(19) United States
(12) Patent Application Publication
(10) Pub. No.: US 2008/0269644 A1
(43) Pub. Date: Oct. 30, 2008

(54) Precision Athletic Aptitude and Performance Data Analysis System

(76) Inventor: Gregory C. Ray, Plano, TX (US)

Correspondence Address: INNOVATION LAW OFFICE OF DENNIS SCHELL, 133 WEST MARKET STREET # 256, INDIANAPOLIS, IN, 46204, US

(21) Appl. No.: 12/110,130

(22) Filed: Apr. 25, 2008

Related U.S. Application Data
(60) Provisional application No. 60/914,308, filed on Apr. 26, 2007.
(60) Provisional application No. 60/947,400, filed on Jun. 30, 2007.

(51) Int. Cl. A61B 5/22

(52) U.S. Cl. 600/587

(57) ABSTRACT

Systems and methods provide collection and analysis of athletic and other human performance and related environmental data. The systems include performance and environmental measurement hardware and data collection and analysis software. Data can be collected in various ways including one of several standardized precision athletic tests. Certification of performance data is provided if environmental data and performance measurement hardware status satisfy preset limits. Performance data can be normalized based on non-standard environmental conditions. The systems and methods establish baseline indications of athletic ability, predict athletic potential for specific sports and team positions, compares individuals to others and to norms, tracks athletic progress, specifies tailored training programs for athletic improvement for specific sports and team positions, provides visual data and analysis, and aids training, physical therapy, and rehabilitation.

### PRECISION ATHLETIC APTITUDE AND PERFORMANCE DATA ANALYSIS SYSTEM COPYRIGHT NOTICE

A portion of the disclosure of this patent document contains material which is subject to copyright protection. The copyright owner has no objection to the facsimile reproduction by anyone of the patent disclosure, as it appears in the US Patent and Trademark Office files or records, but otherwise reserves all copyrights whatsoever.

### BACKGROUND

The present invention relates to instrumentation and data analysis systems, and particularly, to systems for collecting and analyzing athletic and other human performance and related environmental data.

### SUMMARY

The present invention may comprise one or more of the features recited in the attached claims, and/or one or more of the following features and combinations thereof.

The systems and methods provide collection and analysis of athletic and other human performance and related environmental data. The systems include performance and environmental measurement hardware and data collection and analysis software. The system hardware features battery-power, low power consumption, rugged construction, modularity, light weight, rapid setup, compact size, and high portable. Data can be collected in various ways including one of numerous standardized precision athletic tests. Certification of performance data is provided for standardized tests if environmental data and performance measurement hardware status satisfy preset limits. Performance data can be normalized based on non-standard environmental conditions. The systems and methods establish baseline indications of athletic ability, predict athletic performance for specific sports and team positions, compare individuals to others and to norms, track athletic progress, specify tailored training programs for athletic improvement for specific sports and team positions, provide visualized data and analysis, and aid training, physical therapy, and rehabilitation.

One illustrative embodiment of the system includes hardware and software for collecting and analyzing sprint times, for example the total and one or more split times for a 60 yard dash event. Various components of the system communicate with a field device that includes a data processor, for example a portable or handheld computer. The system includes a wireless communication connection, but can also used wired connections. For precise timing measurements, the system includes wireless performance sensors located at various points along the sprint course, for example at the 10 yard, 30 yard, and 60 yard locations. The performance sensors can be, for example, dual laser beams triggered by an athlete interrupting the beam. The system includes locator devices that automatically provide a discrete indication of the accuracy of the placement of the performance sensors. For example, a distance measuring laser and/or GPS device is used to assure that the various sensors are placed within a pre-determined tolerance of 10 yards, 30 yards, and 60 yards from the starting line. The discrete indication may be provided for each separate component of the performance sensors, for example a transmitter, reflector, and/or detector. The discrete indication can be, for example, indicator lights indicating that the location is too close, too far, or correct.

The illustrative embodiment of the system may also include environmental sensors, for example a wind vane and anemometer for determining wind direction, speed, and gusts. The timing and environmental data collected by the processor may be identified as “certified” if certain predetermined parameters are satisfied, for example, the performance sensors are all located within the predetermined distance tolerances and the wind speed and gusts are less than a particular headwind and/or tailwind component for the duration of the event. Certified performance data is only determined automatically by the system hardware and software, may not be manually entered or manipulated, and can then be encrypted and uploaded securely to a remote database, for example via the Internet.

The above and other illustrative embodiments of the system provide for automatic collection and certification of human performance data for other standardized tests, for example, tests for measuring strength, agility, reaction, coordination, speed, power, cardiovascular fitness, or other human abilities and skills.

Illustrative embodiments of the system also provide data collection and analysis of individuals and teams based on demographic, performance, training, and other data. For example, performance data can be normalized based on non-standard environmental conditions or demographics such as age. For example, the system includes data and/or algorithms for analyzing sprint times collected during a high wind condition and determining an approximate sprint time for the same athlete under a no wind condition.

Illustrative embodiments of the system also utilize data and algorithms to establish a baseline indication of athletic performance, to track progress of athletic performance, and to predict future athletic performance for a specific athlete for specific events, tests, or team positions. The system can utilize this data to compare athletes to one another and to statistical norms.

For example, the system includes data and/or algorithms for predicting the athlete's future performance for the specific events. The predication can be based at least in part on demographic, performance, training, and other data associated with the athlete, and data associated with another comparable athlete or data based on statistical analysis of multiple athletes. The prediction can be based solely on an athlete's change in maturity, or alternatively, based on changes in training, body metrics, or other data associated with the athlete. Similarly, the system includes algorithms for predicting athletic potential for specific sports and/or team positions based on data associated with the athlete, and another comparable athlete or data based on statistical analysis of multiple athletes.

One illustrative embodiments of the system for analyzing human performance, includes a data structure for storing image data associated with at least a first person and a second person non-simultaneously performing the same event; and a data processor for analyzing the image data, the data processor adapted for overlying an image of the first person with an image of the second person for at least one particular portion of the event. The image data may be time stamped relative to the beginning of the event and the at least one particular portion of the event is at least one particular elapsed time subsequent the beginning of the event. The image data may include video and the data processor is further adapted for overlying all video images of the first person with all video images of the second person for the duration of the event. The data processor may be further adapted to modify at least one of the image of the first person and the image of the second person for overlying with the respective other image.

### BRIEF DESCRIPTION OF THE DRAWINGS

The detailed description particularly refers to the accompanying figures in which:

FIG. 1 is an illustrative block or state diagram showing software modules of one illustrative embodiment of the system according to the present disclosure;

FIG. 2 is a block or state diagram showing illustrative software modules of the athlete module of FIG. 1;

FIG. 3 is a block or state diagram showing illustrative software modules of the athlete analysis module of FIG. 2;

FIGS. 4A-B are a schematic block diagram showing one illustrative embodiment of the system according to the present disclosure;

FIG. 5 is an perspective view and schematic block diagram of an illustrative portion of the system of FIGS. 4A-B;

FIG. 6 is a perspective view of an illustrative hardware device of the system of FIGS. 4A-B;

FIG. 7 is a perspective view and schematic block diagram of an alternative illustrative embodiment of the system of FIG. 5;

FIG. 8 is a perspective view and schematic block diagram of an alternative illustrative embodiment of the system of FIG. 5;

FIG. 9 is a perspective view and schematic block diagram of an illustrative portion of the system of FIGS. 4A-B;

FIG. 10 is a block diagram of a portion of an illustrative data structure associated with the system of FIGS. 4A-B;

FIGS. 11A-C are plan views for illustrative data entry and/or display associated with the system of FIGS. 4A-B;

FIGS. 12A-E are plan views for illustrative graphical analysis associated with the system of FIGS. 4A-B;

FIGS. 13A-D are plan views for illustrative tabular analysis associated with the system of FIGS. 4A-B;

FIGS. 14A-E are plan views for illustrative tabular display and analysis of an illustrative athlete associated with the system of FIGS. 4A-B;

FIGS. 15A-B are plan views for one set of tabular display and analysis of one illustrative group associated with the illustrative system of FIGS. 4A-B;

FIGS. 16A-B are plan views for another illustrative set of tabular display and analysis of the illustrative group associated with the system of FIGS. 4A-B;

FIGS. 17A-B are plan views for another illustrative set of tabular display and analysis of the illustrative group associated with the system of FIGS. 4A-B;

FIGS. 18A-B are plan views for illustrative tabular display and analysis of illustrative athletes associated with the system of FIGS. 4A-B;

FIG. 19 is a plan view for illustrative video and tabular display and analysis of illustrative athletes associated with the system of FIGS. 4A-B;

FIG. 20 is an illustrative algorithm for performance testing associated with the system of FIGS. 4A-B;

FIG. 21 is an illustrative algorithm for determining data certification associated with the system of FIGS. 4A-B; and

FIG. 22 is an illustrative algorithm for navigation of if device location associated with the system of FIGS. 4A-B.

### DESCRIPTION OF THE ILLUSTRATIVE EMBODIMENTS

For the purposes of promoting and understanding the principals of the invention, reference will now be made to one or more illustrative embodiments illustrated in the drawings and specific language will be used to describe the same.

Human performance as used herein includes athletic performance as well as other human abilities and skills. Examples include basic and complex cognitive and motor skills, such as those that are typically evaluated and impacted by athletic training, physical therapy, health care, and rehabilitation, and other intangibles of human abilities and skills, for example, communication, commitment, attitude, leadership, IQ and work ethic. Performance and other sensors as used herein include passive and active electrical, electro-optical, mechanical, electromechanical, chemical, thermal, acoustic, inertial, radiofrequency, ultrasonic, and other devices that can be used to determine the occurrence of or magnitude of a specific human performance. Examples include discrete and quantitative sensors, including timing devices, electro-optical beam sensors, motion sensors, proximity sensors, and force sensors. Beacon and locator devices as used herein include any passive and active electrical, electro-optical, mechanical, electromechanical, chemical, thermal, acoustic, inertial, radiofrequency, ultrasonic, and other devices that can be used to determine a relative or absolute location or orientation of an item or a displacement or orientation between two items. The locator devices may be capable of determining such location or displacement in one dimension or in multiple dimensions. Examples include GPS devices, active or passive beacons and related detecting devices, and electo-optic beam devices.

Preset parameters as used herein include in values relating to a single or more than one dimension, one value or a range of values, for example a lower and upper limit, and a value and an associated tolerance. Examples include displacement, time, speed, altitude, humidity, temperature, slope, surface friction, surface material, and/or and upper and lower limit thereof. An indicator as used herein includes any device capable of providing a human perceptible output. For example, a light or other visual display, an audible device, or a mechanical actuator. A field device as used herein includes any automated device that can be used at the test or event location for measuring a human performance. For example, a computer, a controller, a processor, and a PDA adapted to detect or measure human performance. A status as used herein includes, the operational state, condition, location, or displacement relative to another device or datum. Sports, events, tests, human performance, and athletes as used herein include those associated with individual and teaming activities, including track and field, football, baseball, basketball, soccer, hockey, Olympic contests, weightlifting, motor sports, water sports, recreational activity, vocational activity, and occupational activity. Algorithms as used herein include, for example, methods, processes, software, and data to perform the described functionality. For example, examples include, include, and variations thereof as used herein are used to introduce an illustrative but not exclusive or limiting list of what can comprise the particular feature described.

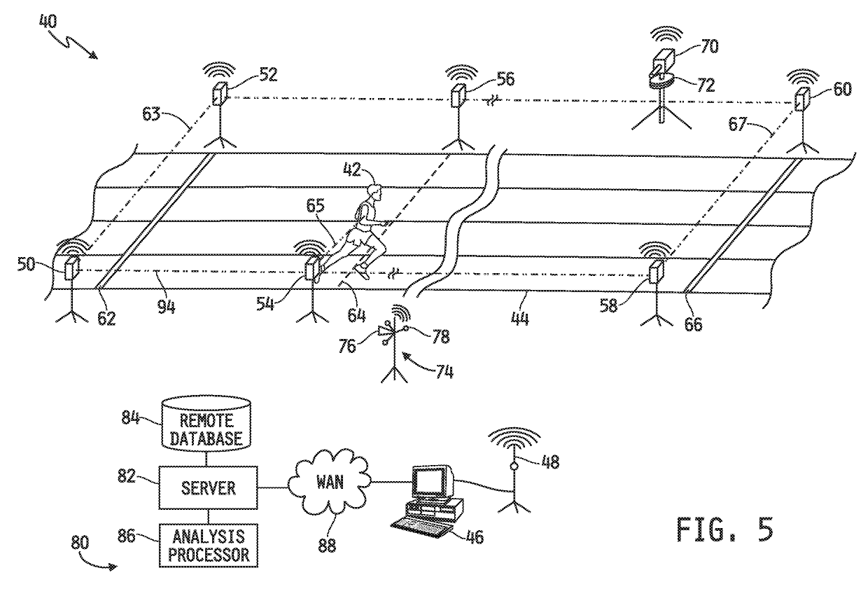

FIG. 5 is a perspective and block diagram view of a system 40 that is one illustrative embodiment for collecting and analyzing sprint times of one or more athlete(s) 42 on a sprint course 44. A data processor 46, for example a portable or handheld computer, provides control and data storage and analysis for the system 40. Communication with other components of the system 40 located at the sprint course 44 are facilitated by a wireless connection 48, for example IEEE 802.11, IEEE 802.16, Bluetooth, cellular, or non-radiofrequency technologies. For precise timing measurements, the system 40 includes performance sensors 50-60 located at various points along the sprint course, for example at the start line 62, the 10 yard line 64, the 30 yard line (not shown), and the 60 yard (finish) line 66. The performance sensors 50-60 may include, for example, dual laser beams providing detection zones 63, 65, and 67, or other sensors capable of detecting one or more athletes 42 advancing across lines 62-66. Performance sensors 50-60 may also include sensors for determining other human performance attributes, for example, wired or wireless heart monitors associated with each athlete 42.

The system 40 also optionally includes one or more video camera(s) 70 or other devices for capturing image data of the athlete 42. Each video camera 70 may include a motor drive 72 and/or a sensor for controller or monitoring the orientation of the video camera 70 relative to the sprint course 44. The system 40 also optionally includes one or more environmental sensor(s) 74, for example, including a wind vane 76 and anemometer 78. Advantageously, the performance sensors 50-60, video cameras 70, and environmental sensors 74 communicate with the processor 46 using the wireless connection 48.

The system 40 also optionally includes a remote resource 80, including, for example, a server 82, remote database 84, and analysis processor 86. The remote resource 80 is located geographically remote from the processor 46 and can be accessed by the processor 46 using a communication link such as a wide area network (WAN) 88, for example the Internet.

The illustrative embodiment of the system 40 may identify collected data, for example, performance data such as timing, as “certified” if certain predetermined parameters are satisfied. For example, the data may be certified if all of the performance sensors 50-60 all located within a predetermined tolerance of the proper locations and the wind speed and gusts are less than a particular headwind and/or tailwind for the duration of the sprint. In order to protect the integrity of certified data, the data may only be automatically determined by the system 40 and may not be manually manipulated. If certified, the data may then be encrypted and uploaded to the remote database 84 for storage and for further comparison and analysis as will be discussed below. The system 40 can also accommodate multiple teams with which various athletes 42 are associated, for example, as is typical at track and field meets.

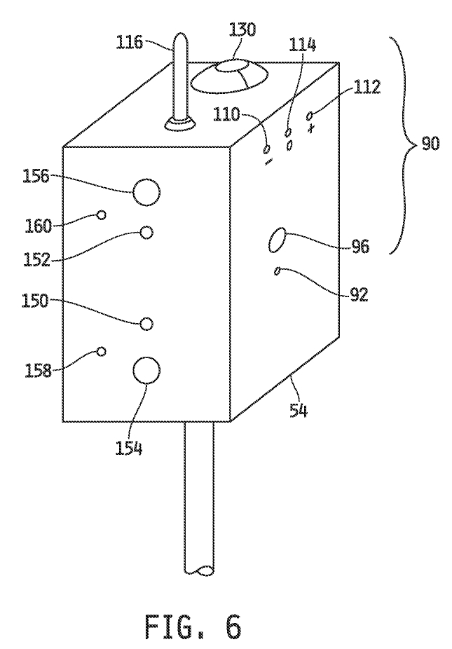

Referring now to FIG. 6, the system 40 includes locator devices 90 that automatically provide a discrete indication of the accuracy of the placement of the performance sensors. For example, a distance measuring laser 92 provides a laser beam 94 (FIG. 5) extending from performance sensor 54 and reflecting off of performance sensor 50 and back to receiver 96. A control circuit (not shown) associated with the laser 92 and receiver 96 determines very precisely the distance between performance sensors 50 and 54. A determination of whether the measure distance is too short, too long, or correct is used to provide an indication of the placement of performance sensor 54. For example, if the distance is too short an indicator 110, for example at a red LED, will be illuminated. If the distance is too long indicator 112, for example a yellow LED, will be illuminated. If the distance is correct, for example equal to 10 yards, an indicator 114, for example a green LED, will be illuminated.

The determination of whether the distance is too short, too long, or correct may be based on a comparison of the measured distance and a preset distance and tolerance, for example 10 yards±0.1 inches. The comparison may be made by the processor 46. For example, the measured distance may be transmitted to the processor 46 using a wireless device 116 associated with the performance sensor 54. Based on the comparison, the processor 46 transmits a signal determining which of the indicators 110-114 is to be illuminated.

Additionally or alternatively, variations of laser-based or other distance and locating devices available in the art may be used to properly locate or to determine the location of performance sensors 50-60, video cameras 70, and environment sensors 74. The location may also or alternatively include, for example, a height or other displacement, an alignment, an orientation, or an absolute or relative geographic position. For example, a laser measuring device could be associated with each of the components 50-60, 70, and 74, or a single two-dimensional or three-dimensional laser measuring system could be used to locate all of the components 50-60, 70, and 74.

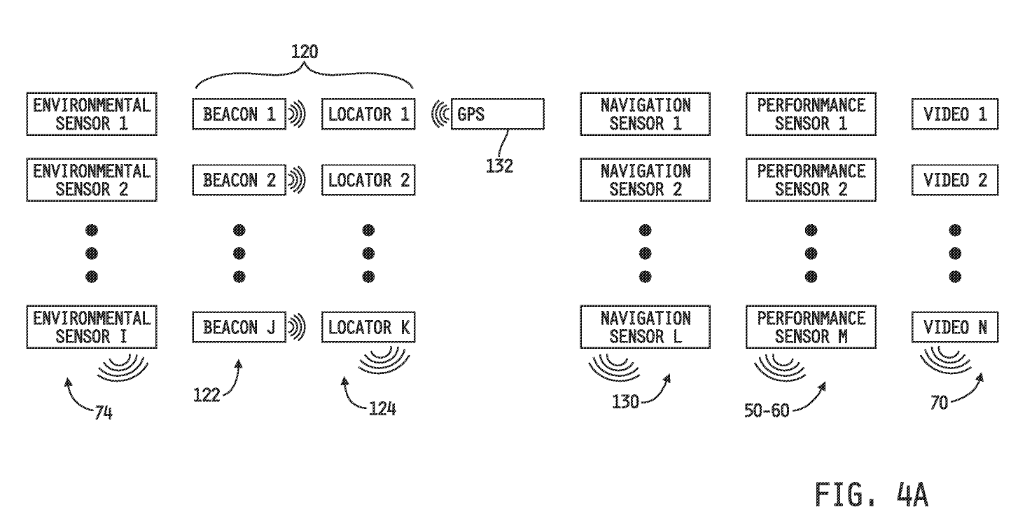

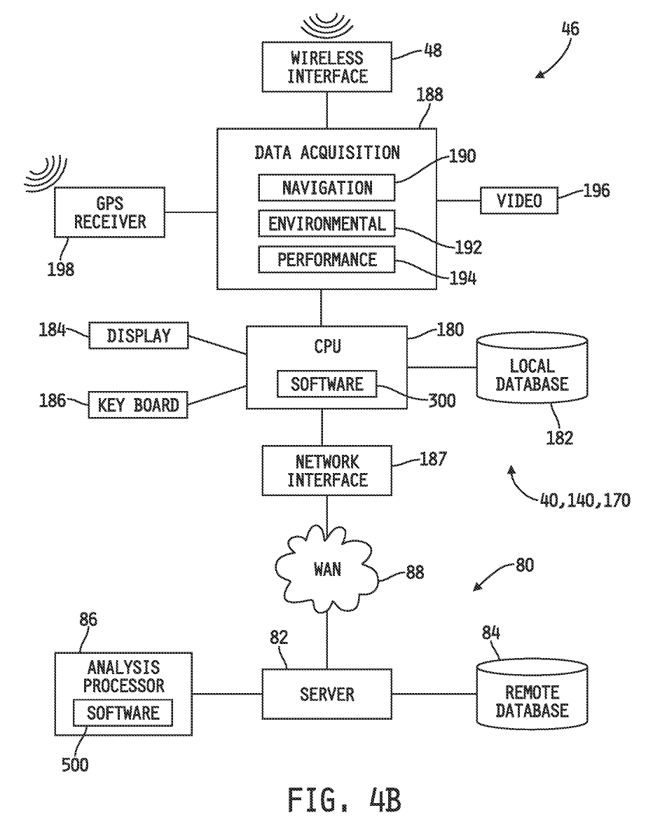

Additionally or alternatively, a passive or active beacon and locator system 120, shown in FIGS. 4A-B and 7, may be used to spatially locate components 50-60, 70, and 74, and even to locate athletes 42 in two or more dimensions. For example, a beacon 122 is associated with the performance sensor 54 and a locator 124 is associated with the performance sensor 150. The beacon 122 may be an active device that transmits a signal that is received by the locator 124. For example, the signal can be electromagnetic, such as a light or radiofrequency signal. Alternatively, both the beacon 122 and locator 124 can be passive devices utilizing ambient electromagnetic energy and image processing. Regardless of the type of beacon 122 and locator 124 used as are known in the art, one or more locators 124 are used to triangulate and locate one or more beacons 122 relative to the locators 124. Similarly, a beacon 126 can be associated with one or more athletes 42 and used with the one or more locators 124 to comprise the performance sensor, thereby replacing or offering a redundant measuring device to the lasers.

Additionally or alternatively, navigation sensors 130 known in the art may be associated with the performance sensors 50-60, the video cameras 70, the environmental sensors 74, and/or the athlete 42 to provide automatic or assisted positioning and locating for the systems 40 and 140. For example, one illustrative navigation sensor 130 utilizes GPS, which is essentially a type of beacon and locator system using satellites as beacons to determine the position of a locator, a ground based GPS receiver. For the systems 40 and 140, differential or carrier phase tracking GPS techniques known in surveying and other applications can be used to precisely determine the relative or absolute position, altitude, and other parameters of the performance sensors 50-60, the video cameras 70, the environmental sensors 74, and even the athlete 42. The navigation sensors 130 may also utilize an additional fixed GPS device 130 as is known in the art in order to achieve an increased precision over standard GPS.

As introduced above, the performance sensors 50-60 may include a discrete detection device such as laser beams to provide detection zones 63, 65, and 67. The detection zones 63, 65, and 67 are used to as triggers for measuring the elapsed time for one or more athletes 42 crossing the starting line 62, the 10 yard split line 64, the 30 yard split line (not shown), and the 60 yard finish line 66. In the illustrative embodiments 40 and 140, each detection zone 63, 65, and 67 include dual laser beams when blocked, as shown for detection zone 65 in FIG. 5, provide a signal to the processor 46. The signal is used by the processor 46 to determine the elapsed time for the athlete 42 to reach the associated distance from the starting line 62, for example the 10 yard split line 64, as is known in the art.

Referring to FIGS. 5 and 6, each pair of performance sensors 50 and 52, 54 and 56, and 58 and 60, can include a laser beam detection circuit. For example, the performance sensor 54 includes two laser emitters 150 and 152, two laser detectors 154 and 156, and two status indicator lights 158 in 160. The corresponding paired performance sensor 56 simply includes a reflective surface for reflecting the laser beams back to the performance sensor 54. Alternatively, the location of the emitters 150 and 152 and detectors 154 and 156 can be distributed differently between performance sensors 54 and 56 to accomplish the same dual beam laser zone 65.

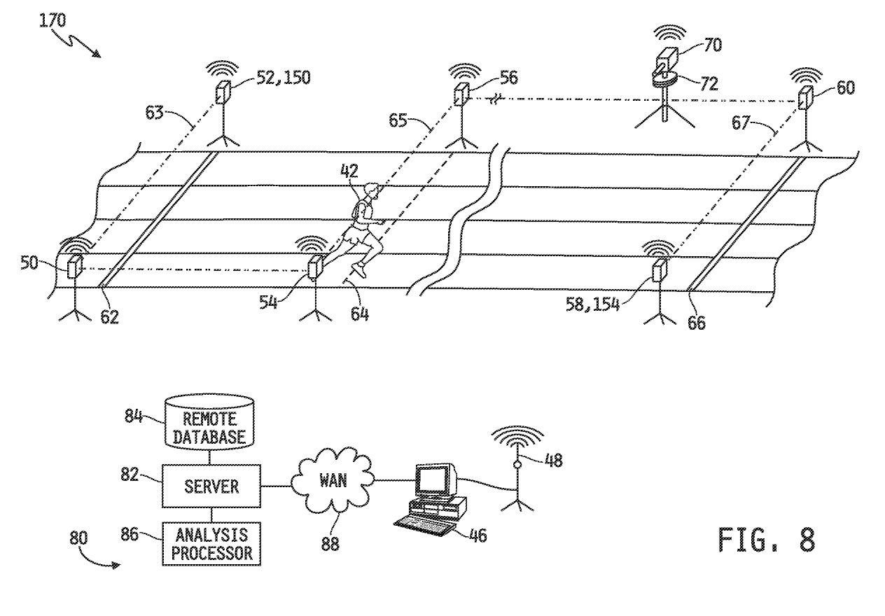

Alternative to the arrangement discussed for FIGS. 5 and 6, as show in FIG. 8, another illustrative system 170 can include in a grid-type laser beam arrangement as is known in the art by using as few is one laser emitter 150, one laser detector 154, and multiple reflectors distributed among the performance sensors 50-60 to provide the same detection and lapsed time determination for the athlete 42 by the processor 46.

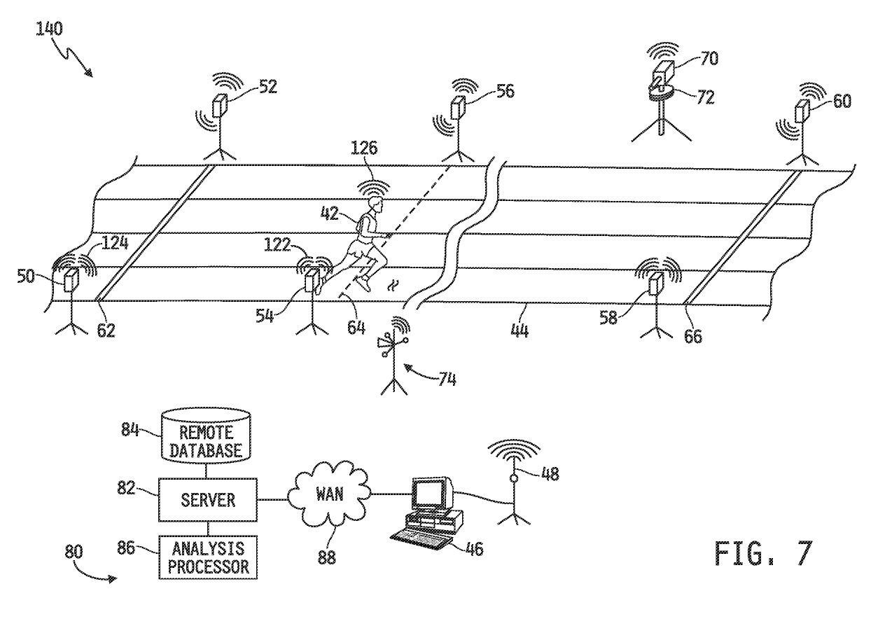

Referring to FIGS. 4A-B, an illustrative block diagram of the illustrative embodiments of the systems 40, 140, and 170, depicts the components show in FIGS. 5, 7, and 8 as well as additional components that may comprise the illustrative systems 40, 140, and 170. For example, the processor 46 includes a CPU 180 and associated precision athletic data analysis (PADA) software 300 for controlling the collection and analysis of performance and other data. The processor 46 may also include a local database 182 so that data may be stored for later transmission to the remote database 84 or for later viewing and analysis. The processor 46 also includes user interface devices, such as a display 184 and a keyboard 186. The display 184 includes a graphical user interface and processor 46 may also incorporate features advantageous to field use, for example a touch sensitive display 184. The processor 46 also includes a wired or wireless communication interface, such as a network interface 187 for accessing the remote resource 80. The analysis processor 86 of the remote resource 80 also includes software 500 for collecting and analyzing the data associated with the local database 182 and/or the remote database 84.

In order to accommodate of specialized signals, instrumentation, and/or processing of the systems 40, 140, and 170, the processor 46 may also include specialized hardware and/or software, for example a data acquisition system 188. Components of the data acquisition system 188 may include, for example, a navigation module 190, an environmental module 192, a performance module 194, a video module 196, and the GPS receiver 198. Each module 190, 192, 194, and 196, respectively facilitates control, data collection, and/or analysis associated with the respective beacon and locator system 120, navigation sensors 130, environmental sensors 74, performance sensors 50-60, and video cameras 70. For example, the navigation module 190 may provide the necessary processing for translation of the beacons 122 relative to the locators 124, or may provide the necessary processing for a high precision implementation of GPS using the navigation sensors 130 and GPS device 132.

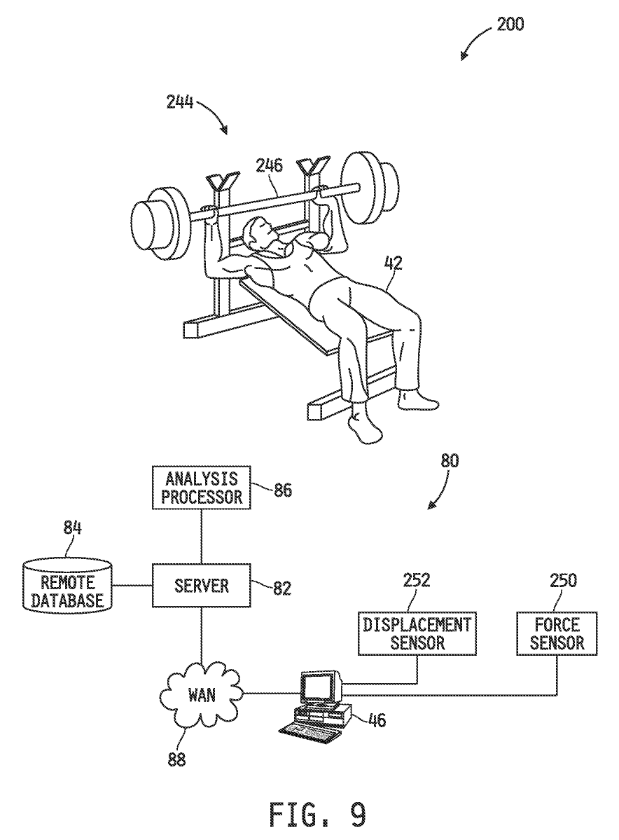

FIG. 9 shows a system 200 that is one illustrative embodiment for collecting and analyzing strength tests of an athlete 242 on a weight apparatus 244. The data processor 46, for example a portable or handheld computer, provides control and data storage and analysis for the system 200. For precise strength measurements, the system 200 includes one or more force sensor(s) 250 and one or more displacement sensor(s) 252 for determining parameters such as force, displacement, velocity, acceleration, and jerk. For example, force sensors 250 may include load, torque, or pressure transducers known in the art, and displacement sensors 252 may include contact, non-contact, and motion sensors known in the art. Alternatively, a single force sensor 250 or a single displacement sensor 252 can be used to determine the displacement, force, and acceleration of the bar 246 as is known in the art.

As with the above illustrative systems 40, 140, and 170, the system 200 also may access remote resource 80, for example using a WAN 88 such as the Internet. The displacement and force data collected by the processor 46 may be identified as “certified” if certain predetermined parameters are satisfied. For example, the sensors 250 and 252 can be adapted and the data received from the sensors 250 and 252 analyzed so that the processor 46 can detect disqualifying events, such as of the bar 246 bouncing off the athlete's chest, a spotter assist, or less than full extension of the athlete's arms. The system 200 may alternatively utilize exercise equipment other than free weights, for example, a resistance or endless-path machine.

The above and other illustrative embodiments of the systems 40, 140, 170, 200 provide for automatic collection and certification of additional human performance data, for example other standardized tests such as tests for measuring strength, tone, agility, reaction, coordination, speed, range of motion, dexterity, balance, gait, perception, and sensation.

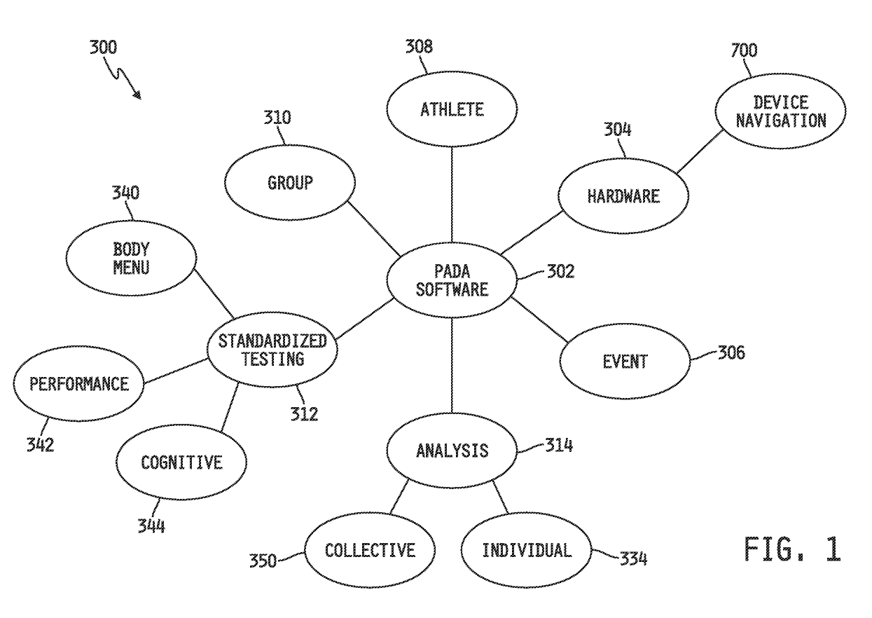

Referring to FIG. 1, a block or state diagram shows illustrative modules of the PADA software 300 associated with the illustrative systems 40, 140, 170, and 200 (hereinafter “system 40”). The modules, including software or algorithms, include a main menu 302, a hardware module 304, an event module 306, an athlete module 308, a group module 310, a standardized testing module 312, and an analysis module 314.

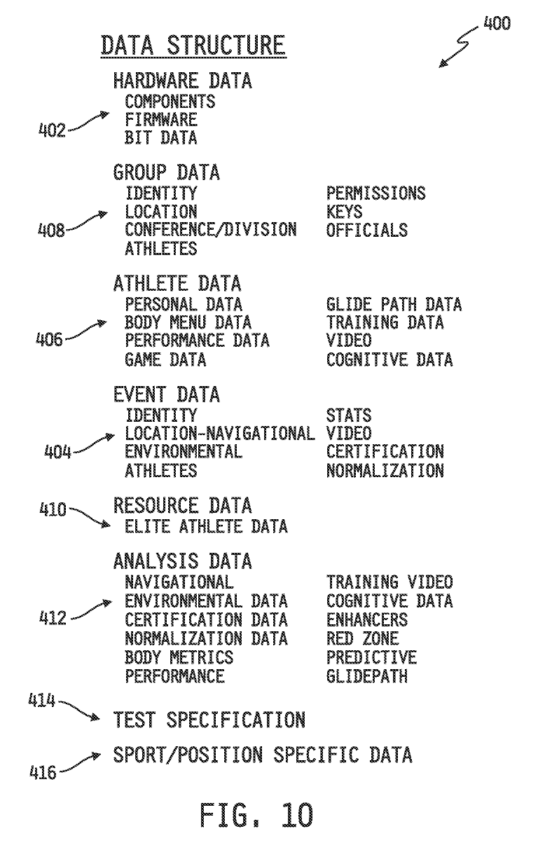

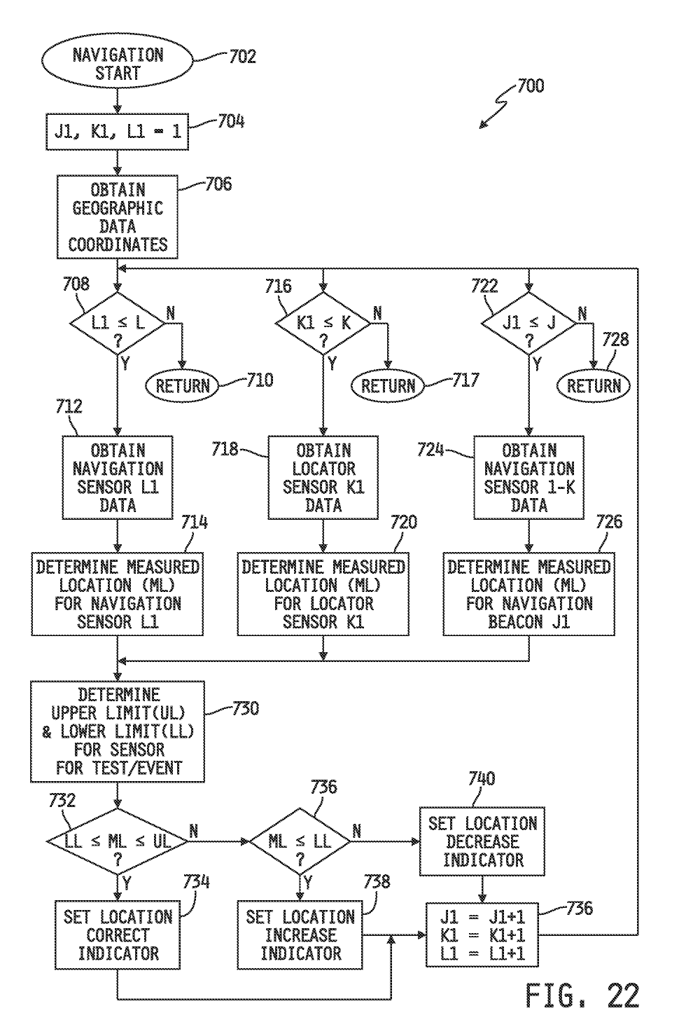

The hardware module 304 includes algorithms associated with the various hardware components of the system 40. For example, the module 304 may include algorithms for auto detecting hardware components, for example those components able to communicate with the processor 46 via the wireless interface 48, algorithms for testing hardware, algorithms for updating the firmware of hardware components, and algorithms for accessing data associated with hardware components, for example hardware data fields 402 of the data structure 400 shown in FIG. 10. The hardware module 304 also includes a device navigation module 700 for determining the position of and/or locating components of the system 40. An illustrative embodiment of the device navigation module 700 is shown in FIG. 22 and that will be further described below.

The event module 306 includes algorithms associated with specific events, including athletic events, specific sports, and standardized tests. The event module 306 include algorithms for determining and monitoring an event identity, athletes associated with an event, statistics associated with an event, video data, normalization of performance data for an event, and certification requirements (preset parameters) for an event, for example required environmental conditions and the proper hardware component location for a selected event, such as the performance sensors 50-60. The event module 306 also includes an algorithm for accessing data associated with events, for example event data fields 404 of the data structure 400 shown in FIG. 10 and which is associated with one or both of databases 84 and 182.

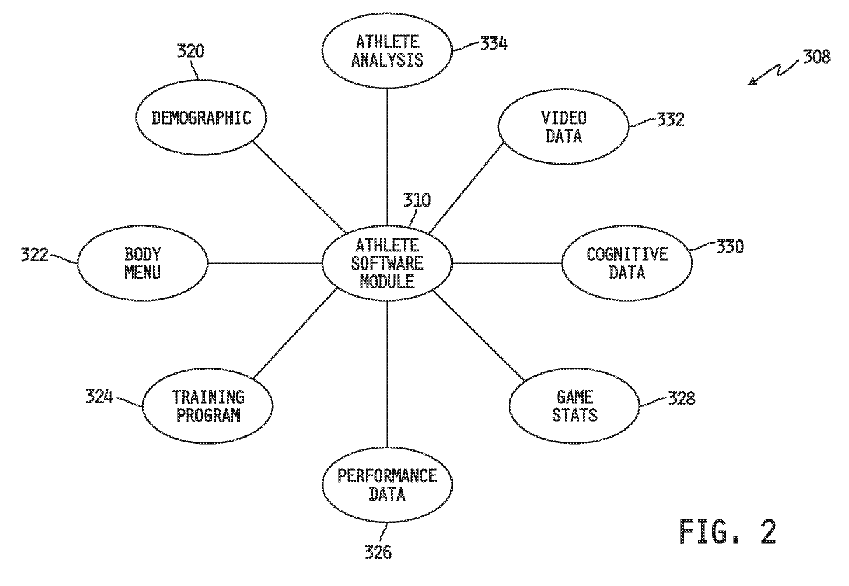

The athlete module 308 is show in additional detail in FIG. 2. The athlete module 308 may include various modules for collecting, displaying, and analyzing or otherwise manipulating data associated with a person, for example an athlete 42, including the athlete data fields 406 of the data structure 400 shown in FIG. 10. Modules include, for example, a main athlete menu 310, a demographic module 320, a body menu module 322, a training program module 324, a performance data module 326, a game statistics module 328, a cognitive data module 330, a video data module 332, and an analysis module 334.

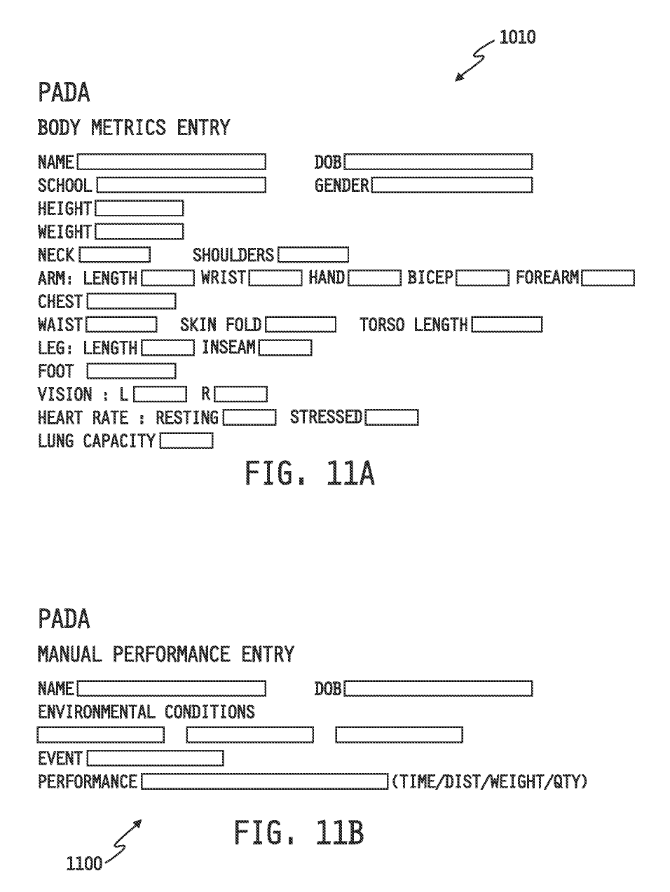

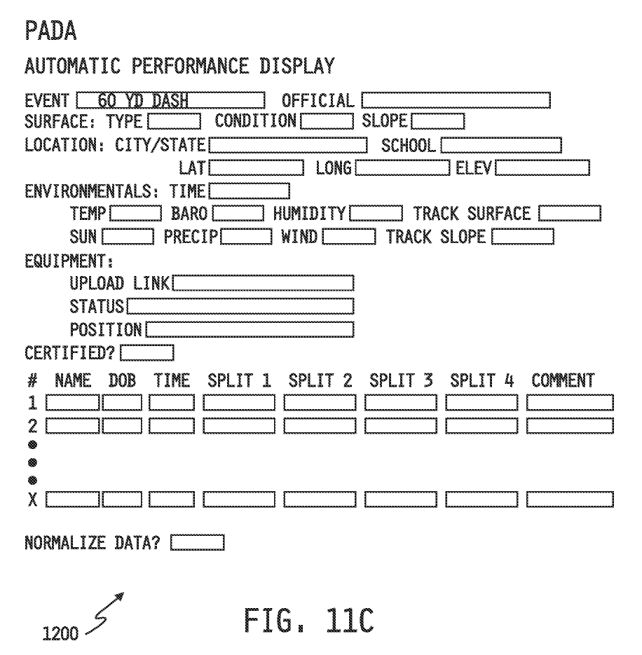

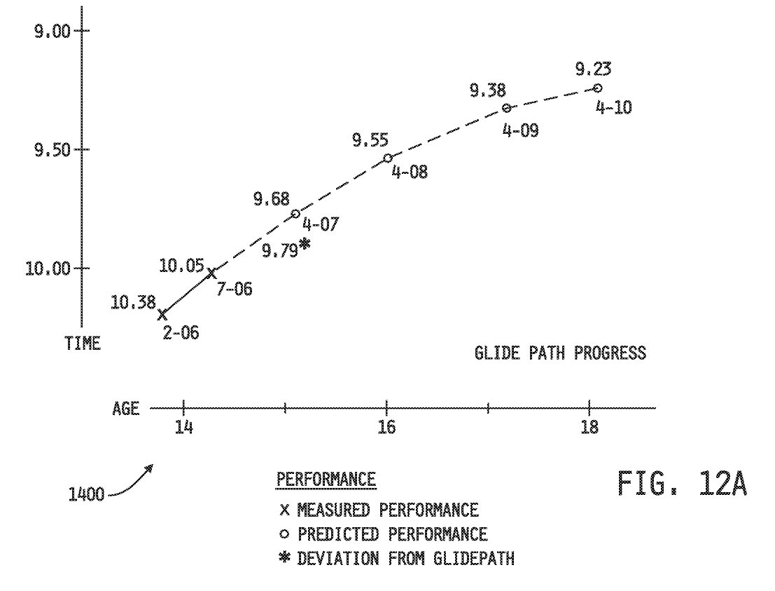

The demographic module 320 includes algorithms relating to general personal data about the athlete 42, including such information as name, gender, date of birth, school, contact information and the like. The body menu module 322 includes algorithms relating to body data (biometrics) for the athlete 42, including such data as height, weight, neck size, shoulder breath, arm length, wrist diameter, hand size, bicep diameter, forearm diameter, chest size, skin fold measurements, body mass index, torso length, leg length, inseam length, foot size, uncorrected and corrected visual acuity, resting and stressed heart rate, lung capacity, bone dimensions, and other such biometrics. The body data may be manually entered and/or obtain automatically and certified using measurement sensors associated with the system 40. For example, referring to FIG. 11A, the body metric entry form 1010 can be used to enter data for the athlete 42. Other data, for example, cognitive data can be entered in a similar fashion if hardware for automatic testing is not available in a particular configuration of the system 40. The body menu module 322 includes algorithms for analyzing a set of the body data in order to categorize the athlete 42 in a particular “body menu”.

The training program module 324 includes algorithms associated with training data for the athlete 42, including such data as diet, sleep, and training regime, or for a group of athletes. The training regime data may include many aspects of a training program, including the particular activities, durations, intensities, and schedules. The performance data module 326 includes algorithms associated with performance data for an athlete 42, specifically measures of human performance, for example, the data collected and/or determined by the system 40, including the performance sensors 50-60.

The game statistics module 328 includes algorithms associated with game statistics, for example, overall team statistics for a particular game or other event, and individual statistics for the athlete 42, for a particular game or other event. The cognitive data module 330 includes algorithms associated with cognitive data for an athlete 42, including psychometrics. The video data module 332 includes algorithms associated with image data for an athlete 42. The athlete or individual analysis module 334 includes algorithms associated with data analysis directed to an individual rather than a collective such as a group or team. The athlete analysis module 334 will be discussed in more detail below in the description of the analysis module 314.

Referring again to FIG. 1, the group module 310 includes algorithms associated with specific groups, including athletic teams or other collectives as selected based upon group data fields 408 (FIG. 10), event data fields 404, or athlete data fields 406. The group module 310 includes algorithms for accessing and manipulating the group data 408, including data such as identity, city, school, conference, division, associated athletes and officials, teams, and system information such as the permissions and encryption keys associated with a particular group.

The standardized testing module 312 includes algorithms associated with obtaining and manipulating data for an athlete or group, including a body menu testing module 340, performance testing module 342, and cognitive testing module 344. Generally performance data is automatically collected by the system 40; however, referring to FIG. 11B, a manual performance entry form 1100 can be used for manually entering performance, environmental, or other data for a test or event for which automatic collection hardware is not available.

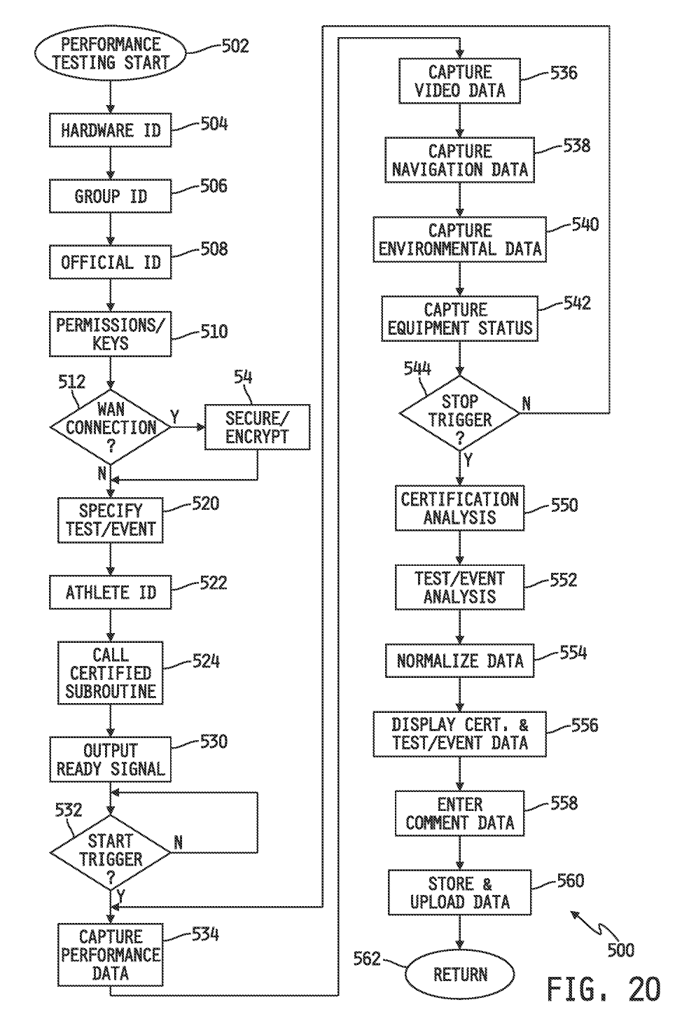

Referring now to FIG. 20, a flowchart of an illustrative algorithm 500 of the performance testing module 342 is provided. All or portions of the algorithm 500 may be executed by one or more of the components of the illustrative systems 40, 140, 170, and 200, including the processor 46, the analysis processor 86, performance sensors 50-60, environmental sensors 74, beacon and locator system 120, navigation sensors 130, and video cameras 70; however, to describe only one illustrative embodiment and for brevity, references to executing the algorithm 500 will only be made to the processor 46 and the system 40.

When called by the testing module 312, execution of the algorithm 500 by the processor 46 begins at step 502. In step 504, the processor 46 obtains hardware data relating to the system 40, for example from the hardware data fields 402 of the data structure 400 and/or from auto-detection of available hardware in communication with the processor 46. In step 504, the processor 46 may also execute other hardware related functions, for example a built-in test of various hardware components of the system 46.

In step 506, the processor 46 obtains data relating to the group, if applicable. For example, the processor 46 can obtain the identity and associated permissions and keys licensed for the system 40 from the group data fields 408 of the data structure 400. Additionally or alternatively, the processor 46 may receive manual input relating to the group data, for example from the keyboard 186, or another input device such as a magnetic card or other security device. In step 508, the processor obtains data relating to a group or event official, if applicable. For example, in step 508, identity of an official entered via the keyboard 186 may be verified with data stored in the group data field 408 of the data structure 400, or verified with data stored in the remote database 84. In step 510, the processor 46 determines the permissions and keys authorized by the combination of the group and official. For example, permissions may include which events are licensed and/or authorized, and the key may include encryption keys for communicating data with the remote resource 80.

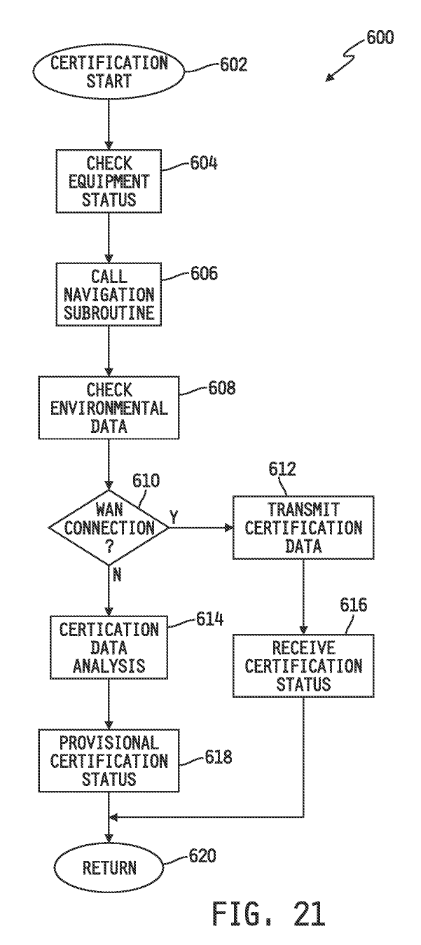

In step 512, the processor 46 determines whether a network connection is available with the remote resource 80. If so, in step 514, secure, encrypted communication will be established between the processor 46 and the remote resource 80. In step 520, the processor 46 determines the standardized test or other event for which data will be collected. For example, the official may select the test from a menu or the processor 46 may automatically determine the type of test based on the available hardware components of the system 40 in communication with the processor 46. In step 522, the processor 46 determines the athlete(s) 42 for which performance data will be collected. Athlete(s) 42 may be entered by the official, or automatically detected by the processor 46 if uniquely identifying beacons 122 are utilized with the system 46. In step 524, the processor 46 calls the certification algorithm 600 shown in FIG. 21.

The certification algorithm 600 begins at step 602. In step 604, the processor 46 determines the status of the hardware components required to conduct the selected test, for example the performance sensors 50-60. The status may be determined by evaluating the results of a built-in test conducted in step 504 of the performance testing algorithm 500, or by presently polling the hardware components. In step 606, the processor 46 calls the navigation algorithm 700, shown in FIG. 22.

The navigation algorithm 700 begins at step 702. In step 704, the processor 46 initializes variables associated with navigation hardware, for example, J is the number of beacons 122 and J1=1, K is the number of locators 124 and K1=1, and L is the number of navigation sensors 130 and L1=1. In step 706 the processor 46 obtains a reference datum, for example the geographic coordinates and altitude of the processor 46 or a component of the system 40, for example one or more of the performance sensors 50-60 such as the performance sensor 50 located at the starting line 62. For example, the reference datum can be determined by the GPS receiver 198, or a navigation sensor 130, for example one associated with the performance sensor 50. The reference data may be stored with the event data and/or used in determining the location of other components of the system 40, including performance sensors 50-60, video cameras 70, environmental sensors 74, beacons 122, locators 124, and navigation sensors 130.

Steps 708, 716, and 722 may all be executed by the processor 46, or a subset of the steps 708, 716, and 722 may be selected depending on the hardware available for and selected for navigation. In step 708, the processor 46 determines whether a navigation sensor 130 numbered L1 needs to be located. If so, the algorithm 700 continues at step 712, else in step 710, the processor 46 returns execution to the calling algorithm 600. In step 712, the processor 46 obtains the navigation data from the navigation sensor L1 130. In step 714, the processor 46 determines the measured location ML for the navigation sensor L1 130.

In step 730, the processor 46 determines the upper limit UL and the lower limit LL for the navigation sensor L1 130 or other device being navigated. For example, the upper limit you well and the lower limit LL may be obtained from the event data field 404 of the data structure 400. In step 732, the processor 46 determines whether the measured location ML is between the upper limit UL and the lower limit LL. If so, the algorithm 700 continues at step 734, else step 736 is executed. At step 734, the processor 46 sets the indicator 114 (FIG. 6) indicating the location of the navigation sensor L1 130, for example associated with the performance sensor 54, is properly positioned.

If the measured location ML was not determined to be between the lower limit LL and the upper limit UL, in step 736, the processor 46 determines whether the measured location ML is less than the lower limit LL. If so, execution of the algorithm 700 continues at step 738. At step 738, the processor 46 sets an indicator 110 indicating that the location is too close to the reference datum, for example the performance sensor 54 needs to be moved further down the sprint course 44 and away from the performance sensor 50. If in step 736 it is determined that the measured location ML is not less than the lower limit LL, then in step 740, the processor 46 sets the location indicator 112 indicating that the location is too far from the reference datum, for example the performance sensor 54 needs to be moved further toward the performance sensor 50.

After execution of step 734, 738, or 740, in step 736, the processor 46 sequences to the next one of J1, K1, and L1. Execution of the algorithm 700 and continues at one or more of step 708, 716, and 722. If locators 124 need to be navigated, in step 716, the processor determines whether a locator 124 numbered K1 needs to be located. If so, the execution of algorithm 700 continues at step 718, else at step 717 the execution returns to the calling algorithm 600. In step 718, the processor 46 obtains the navigation data for the locator K1 124. In step 720, the processor 46 determines the measured location ML for the locator K1 124. In step 730, execution of algorithm 700 continues as discussed above.

If beacons 122 need to be navigated, in step 722, the processor determines whether a beacon 122 numbered J1 needs to be located. If so, the execution of algorithm 700 continues at step 724, else at step 728 the execution returns to the calling algorithm 600. In step 724, the processor 46 obtains the navigation data for beacon J1 122 from each of the locators 1-K 124. In step 726, the processor 46 determines the measured location ML for the beacon J1 122, for example, by geometric triangulation using the navigation data obtained from each of the locators 1-K 124. In step 730, execution of algorithm 700 continues as discussed above.

The execution of the algorithm 700 continues until all of the navigation sensors 130, beacons 122, and locators 124 that need to be navigated have been properly located, then execution returns to the calling algorithm 600.

Referring again to FIG. 21, execution of algorithm 600 continues at step 608. In step 608, the processor 46 obtains and evaluates environmental data. For example, the processor 46 obtains data from each of the environmental sensors 74 and stores, compares, manipulates, and/or displays the environmental data. For example, the processor 46 may obtain preset environmental parameters from the event data fields 404 of the data structure 400 and provide an indication of whether the current environmental data satisfies the preset environmental parameters, for example, that required for certification. Environmental data may include, for example, characteristics of the atmosphere, geography, event surface and orientation, and testing equipment.

In step 610, the processor 46 determines whether a communication connection with the remote resource 80 is available. If so, execution of the algorithm 600 continues at step 612, else step 614 is executed. In step 612, the processor 46 transmits the data required for certification of the performance data to be collected. For example, the equipment status, navigation, and environmental data can be transmitted to the remote resource 80 in order for the analysis processor 86 to determine whether the preset parameters are satisfied to provide certification of the performance data to be collected. In step 616, the processor 46 receives the certification status from the remote resource 80.

If in step 610, the processor 46 determines that a communication connection to the remote resource 80 was not available, then in step 614, the processor 46 will complete the certification analysis of the data, for example the equipment status, navigation, and environmental data and the associated preset parameters. In step 318, processor 46 will set a provisional certification status for the performance data if the preset parameters were satisfied in step 614. After execution of step 616 or 618, in step 620 execution returns to the calling algorithm 500.

Referring again to FIG. 20, execution of algorithm 500 continues at step 530. Step 530, the processor 46 provides a ready signal indicated that the system 40 is prepared to collect performance data for the specified evidence. For example, the ready status may be indicated on the display 184. In step 532, the processor 46 waits until a starting trigger is received indicating that performance and other data should be collected by the processor 46. For example, the trigger may be manually initiated, for example via the keyboard 186, or may be automatically triggered, for example using a sound sensor (not shown) adapted to be triggered by the sound of a starting gun.

In step 534, the processor 46 captures performance data from the performance sensors 50-60. In step 534, the processor 46 also provides any monitoring and control necessary for the proper functioning of the performance sensors 50-60. In step 536, the processor 46 captures video data from the video cameras 70 and also provides any monitoring and control necessary for the proper functioning of the video cameras 70. For example, the video camera 70 may be panned to keep athletes 42 in view using motor 72 and based on image processing and tracking algorithms known in the art.

In step 538, the processor 46 captures navigation data associated with selected components of the system 40 for which location monitoring is desired during the event and optionally the athletes 42. In step 540, the processor 46 captures environmental data from selected environmental sensors 74 for which monitoring is desired during the event, for example wind conditions. In step 544, the processor 46 determines whether a stop trigger signal has been received, for example all athletes 42 breaking the detection zone 67 located at the finish line 66. If so, the execution of algorithm 500 continues at step 550, else execution loops to step 534.

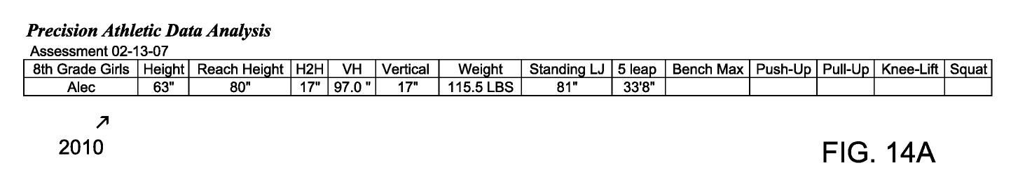

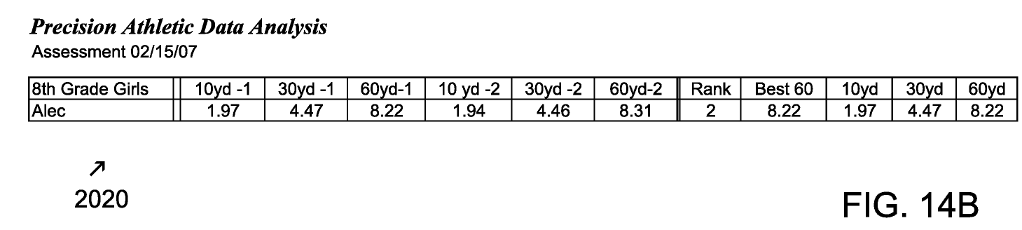

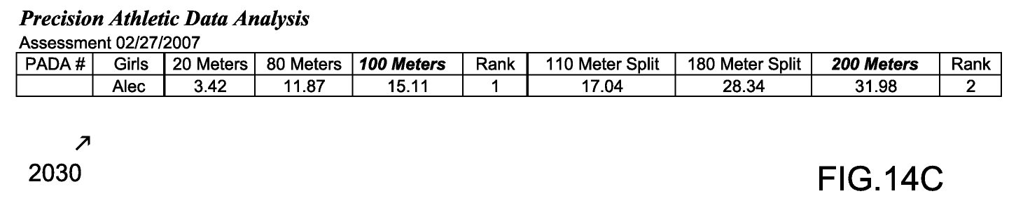

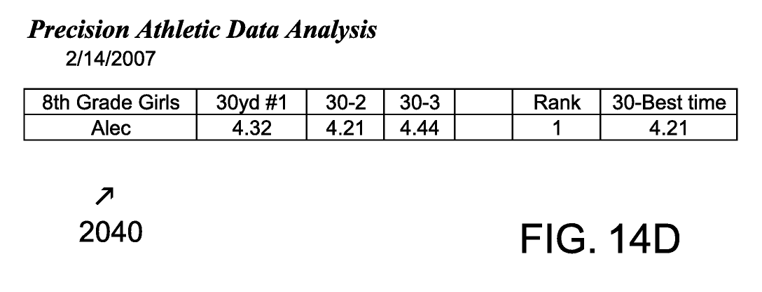

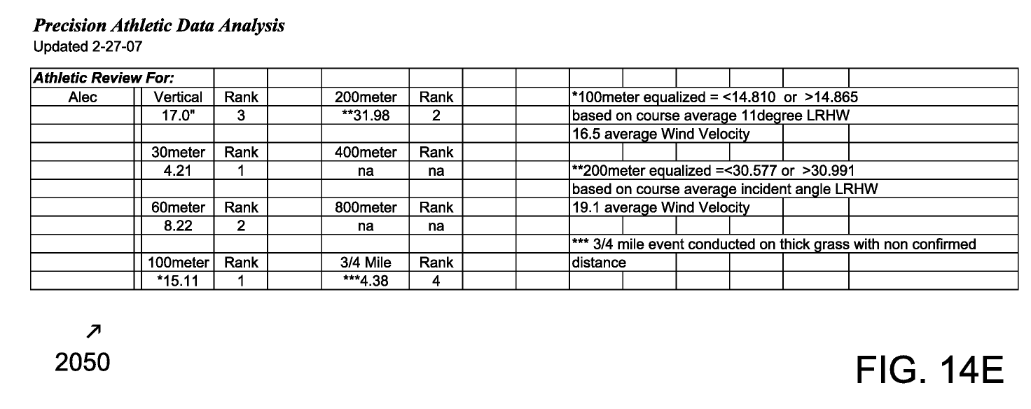

In step 550, the processor 46 can again verify the certification of the performance data collected further based on the data collected during the event and/or collected immediately subsequent to the event. For example, in step 550, the processor 46 can again call the certification algorithm 600 shown in FIG. 21. In step 52, the processor 46 can analyze various aspects of the data collected for the test or other event. For example, ranking of the athletes 42 and other such analysis as will be further discussed below for FIGS. 14A-19.

In step 554, the processor 46 can normalize the performance data collected in nonstandard conditions. For example, a second set of performance data can be calculated that compensates for wind conditions and provides a prediction of an athlete's performance in a no wind condition. In step 556, the processor 46 displays the various test or event data, including the performance data, certification status, and any normalized data. The display may also include generation of onscreen or paper reports that include tabular, graphical, or a combination of presentations. For example, the illustrative performance display 1200 shown in FIG. 11C. Other illustrative examples include FIGS. 14A-19.

In step 558, the processor 46 provides an opportunity to enter manual or comment data associated with the test or event. For example, system 46 secures and prevents manual manipulation of automatically collected data; however, particular data may be manually entered. For example, rather than using automated environmental sensors to determine the sun illumination and precipitation, such data can be manually entered by the official. Additionally, comment fields may be associated with collected data that cannot be manually manipulated in order to indicate and communicate important information, for example an athlete 42 suffering an injury during and not completing a sprint event. In step 560, data associated with the test or event is stored, for example in the local database 22, and can be securely uploaded to the remote database 84. In step 562, the processor 46 returns execution to the calling algorithm, for example the standardized testing module 312.

Referring again to FIG. 1, the PADA software 300 also includes an analysis module 314. The analysis module 314 includes an individual analysis module 334 having algorithms for analyzing specific individuals such as a specific athlete 42, and a collective analysis module 350 having algorithms for analyzing groups of individuals, for example a sports team. The discussion of the illustrative analysis module will primarily focus on the individual analysis module 334; however, the features and concepts are equally applicable to the collective analysis module 350 for analyzing an athletic team or other group and identify glidepaths, training changes, scenarios, predicted performance and the like for a group and team performance rather than an individual. The analysis module 314 includes algorithms for analyzing and providing data as is discussed further below as well as elsewhere in this disclosure and in the various drawings illustrating the disclosure. The analysis module 314 may also include other human performance and image analysis algorithms known in the art but disclosed with specificity herein.

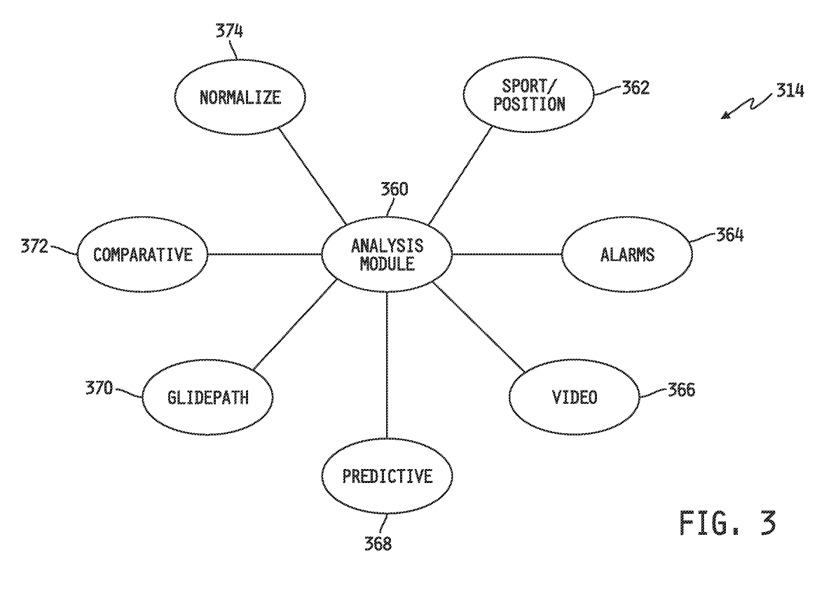

Referring to FIG. 3, the illustrative analysis module 314 includes a main menu 360, a sport or team position analysis module 362, an alarm module 364, a video analysis module 366, a predictive analysis module 368, a glide path analysis module 370, a comparative analysis module 372, and a normalization module 374. The normalization analysis module 374 includes algorithms that compute an additional set of performance data for one or more athletes 42 to compensate for nonstandard environmental conditions. For example, for an event conducted on a windy day or on a soft or turf surface rather than a hard surface, the algorithms can compute revised performance data that predicts the performance of athletes 42 had the event and conducted under standard and environmental conditions.

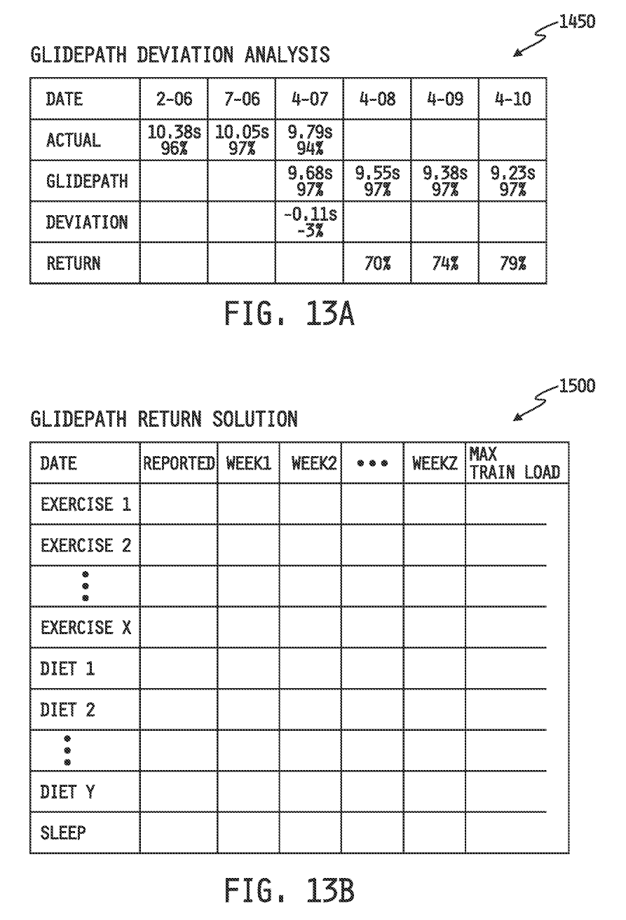

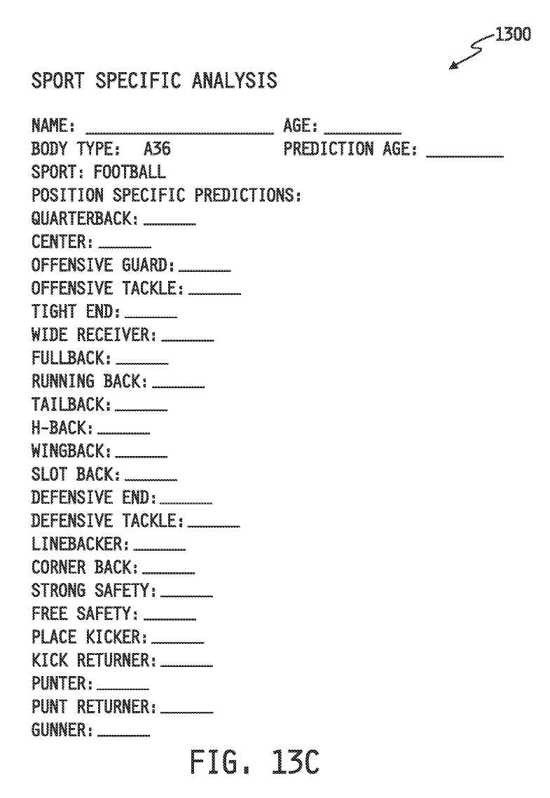

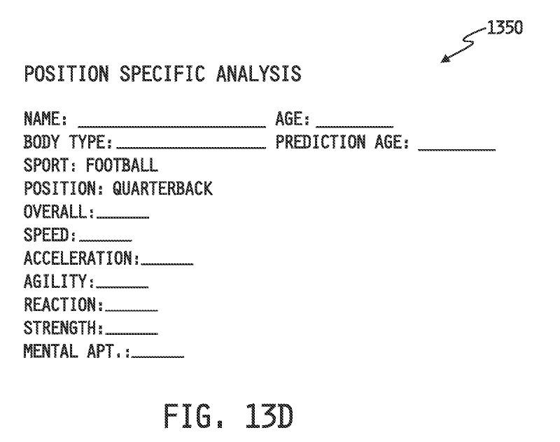

The sport and team position analysis module 362 includes algorithms that predict present or future performance of a particular athlete 42 for specific sports, and specific team positions. For example, as shown in FIG. 13C, sport specific analysis 1300 provides quantitative predictions for an athlete 42. The quantitative predictions may be provided for each of a number of different team specific positions. Additionally, referring to FIG. 13D, for a specific one of the team positions, for example quarterback as shown in the position specific analysis 1350, the analysis module 362 can provide the data upon which the analysis was based as well as specific quantitative predictions particularly associated with the specific position. The algorithm associated with the sport and team position specific module 362 utilizes demographic, body menu, performance, cognitive, and other data associated with a particular athlete 42. The algorithm can further utilize data associated with other athletes, for example resource data 410 from the data structure 400. For example, the algorithm may select specific resource data 410, for example performance and other data of an elite athlete for the specific team position, based on similarity of body menu, performance and other data of the athlete 42 and a selected elite athlete. For example, if the resource data 410 includes body menu, performance, game stats, and other data for the elite athlete over a period of time, for example during the time the elite athlete was maturing, the algorithm can use the data to predict future performance, including game stats, of a substantially similar athlete 42 been analyzed. Similarly, the algorithm can utilize a statistical computation of a selected group of athlete, for example, NCAA athletes, professional league athletes, top professional athletes, and the like. Additionally or alternatively, the algorithms can provide specific training recommendations specific to the data associated with the athlete 42 and the team specific position.

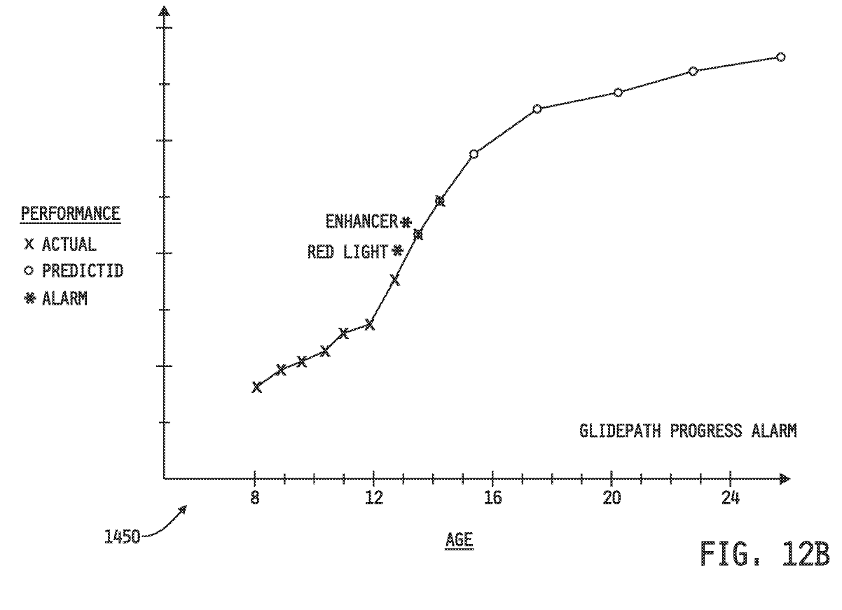

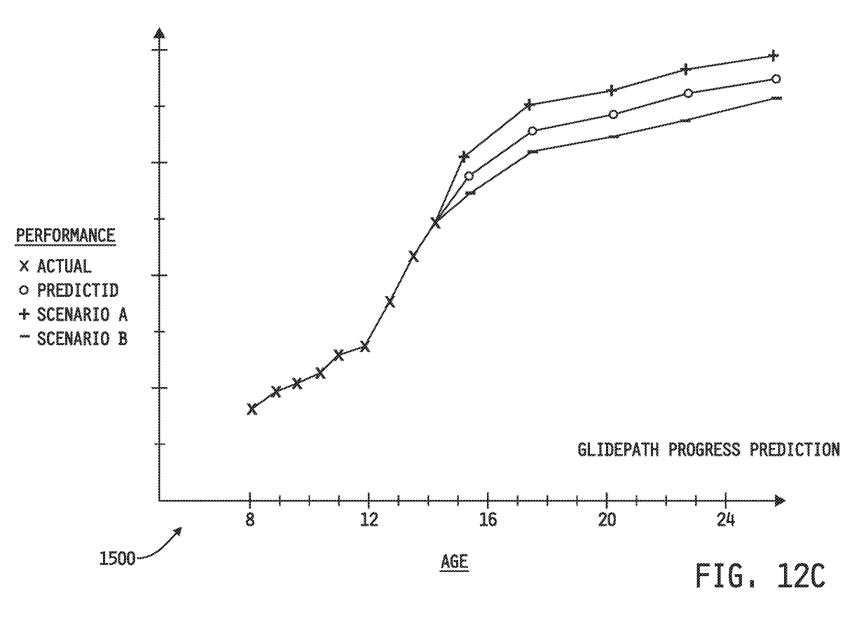

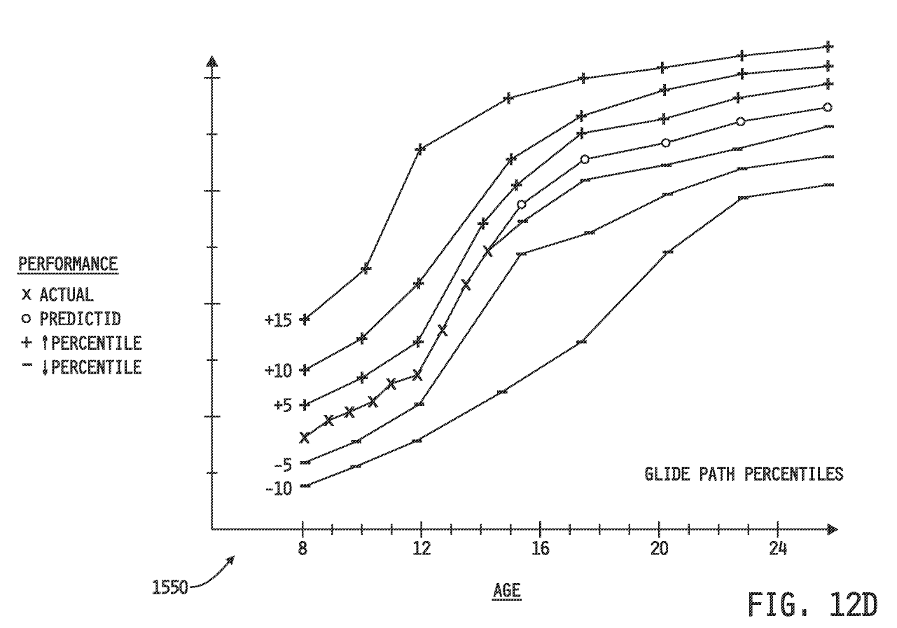

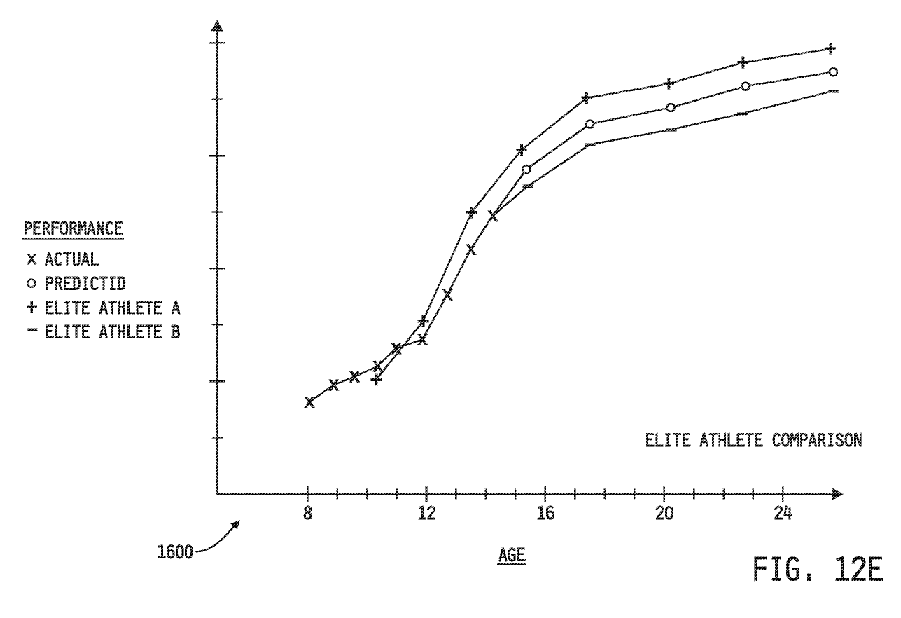

Referring to FIGS. 12A and 13A, glidepath analysis module 370 provide algorithms for determining predicted performance of an athlete 42 and displaying the associated data in a graphically format, as shown for glide path progress graph 1400 in FIG. 12A, in a tabular fashion, showing for glide path deviation analysis table 1450 in FIG. 13A, or in a combined tabular and graphical fashion (not shown). The algorithms of the glidepath analysis module 370 uses performance and other data of athlete 42, for example a performance test on 2-2006 and 7-2006 to provide a projection of the likely performance over a period of time, for example 4-2007 through 4-2010 as shown for graph 1400. The algorithms may also use resource data 410 in order to provide the predicted glidepath as the athlete 42 continues to train, mature, and develop. For example, the algorithm may determine and use a statistical computation based on a number of athletes having similar body menu and other data, or may use the data of a specific elite athlete with which the athlete 42 has a similar body menu and/or other selected data. Additionally and alternatively, all features of the glidepath analysis module 370 also provides algorithms for predictions of data other than performance data, for example body menu data such as height or sport or team specific performance.

Referring again to FIGS. 12A and 13A, the glidepath analysis module 370 also includes algorithms to monitor the athlete's progress. For example, a deviation from the predicted glidepath, for example the performance time of 9.79 seconds measured on 4-2007 is shown in FIG. 12 way as an asterisk deviating below the predicted glidepath. Similarly, as shown in FIG. 12, the deviation and statistical information regarding it is indicated. The glidepath analysis module 370 may also include algorithms to provide a change in or other characteristics of a training program so that the athlete 42 may achieve the predicted glidepath over a period of time, including a statistical likelihood of achieving the glidepath or other goal. For example, the glidepath returned solution table 1500 provides reported, recommended, and maximum training program attributes, including specific exercises in diet features, and sleep. The glidepath analysis module 370 also allows the glidepath to be updated when desired based on additional data, forming a new predicted glidepath.

Referring to FIG. 12B, the alarm analysis module 364 includes algorithms for determining an alarm condition and based on a sudden or statistically unlikely increase in performance based on past performance and other data for the athlete 42. For example, the red light alarm as shown in glidepath progress alarm graph 1450 can provide the athlete and coach an indication that the athlete is performing above or well above the previously projected glidepath. Additionally, the enhancer alarm can warn the coach and official that a statistically unlikely increase in performance for the athlete 42 has occurred based on past performance and other data, the is alerting them to consider the possibility that the athlete 42 is utilizing performance enhancement supplements or drugs that may be unlawful or otherwise not acceptable.

Referring to FIG. 12C, the predictive analysis module 368 includes algorithms that provide future performance scenarios. For example, said scenario A and scenario B may be entered as possible changes to attributes of an athlete's 42 training program. Based on the glidepath data for athlete 42 in the entered scenarios, the algorithms provide glidepath performance prediction graph 1500 indicating predicted changes in performance from the earlier determine glidepath for scenario A and scenario B. Additionally and alternatively, scenarios may be based on changes in any other data, for example the athlete 42 growing two inches, gaining 20 lbs, or gaining 5% strength over the next nine months.

Referring to FIG. 12D, the comparative analysis module 472 includes algorithms for comparing measured and predicted glidepath data with other athletes, including on a statistical percentile basis as shown in glidepath percentile comparison graph 1550. Similarly, the comparative analysis module for 72 also includes algorithms for comparing one or more specific athletes with selected athlete 42, for example elite athlete A and elite athlete B as shown in the athlete comparison graph 1600 in FIG. 12E.

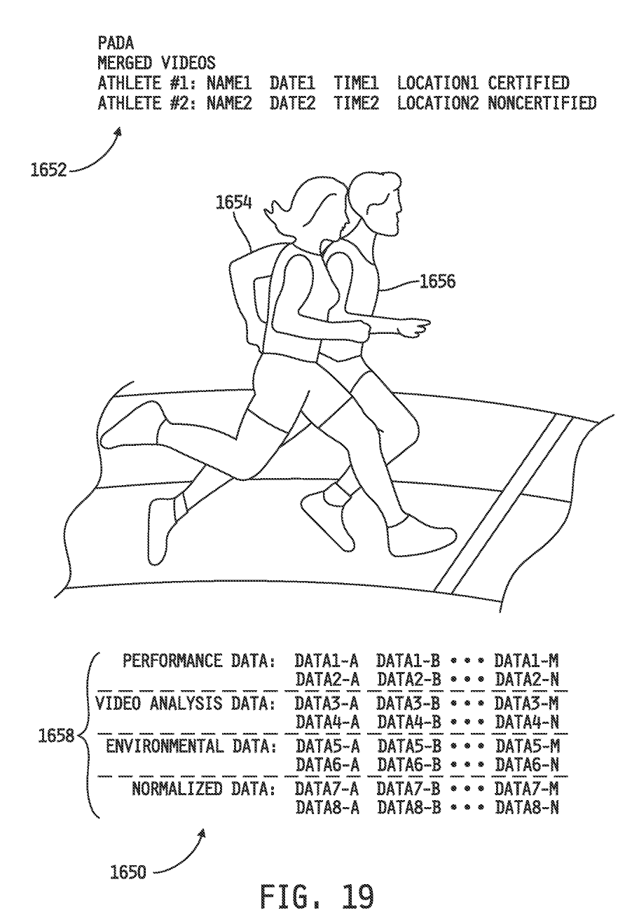

Referring to FIG. 19, the video analysis module 366 provides algorithms for analyzing collected image data. For example, video analysis display 1650 includes tabular data 1652 providing demographic and other data for a first athlete 1654 and a second athlete 1656, and tabular data 1658 for athlete's 1654 and 1656, for example performance, environmental, normalized, and video analysis data. For example, the algorithms of video analysis module 362 may utilize image processing and analysis data and algorithms known in the art in order to additional performance and other characteristic data of the athletes 1654 and 1656. For example, the algorithms may provide analysis of the athlete's style, form, stride length, foot strike, pronation, and the like. Additionally, the algorithms can provide merging of two separate image data sets. For example, while video and other data for athlete 1654 and 1656 may have been collected for a 60 yard dash, the actual collected data may have occurred during different testing sessions. Using the time or spatial data associated with the video images for athlete 1654 and 1656, a merged video data set can be produced that over lays the image of one of the athletes 1654 and 1656 over the video data set for the other athlete. Additionally, for comparing athletes 1654 and 1656, performance data can be normalized based on age or other differences.

The illustrative system 40 and associated software 300 include algorithms for determining and presenting various data and analysis reports, an illustrative set of which will now be described. An individual assessment summary report 2010 shown in FIG. 14A includes demographic, body menu, and performance data for a selected athlete 42. Similarly, a first individual event analysis report 2020 shown in FIG. 14B includes demographic, performance, and analysis data for a selected athlete 42. Specifically, report 2020 includes split and final 60 yard dash times for multiple tests and an analysis ranking of the athlete 42 for the 60 yard dash event among a selected peer group. Similarly, a second individual sprint analysis report 2030 shown in FIG. 14C includes split and final 100 meter and 200 meter dash times as well as an analysis ranking of the athlete 42 for each of the events among a selected peer group. Similarly, a third individual sprint analysis report 2040 shown in FIG. 14D includes 30 yard dash times for multiple tests for that event as well as an analysis ranking of the athlete 42 among a selected peer group based on the athlete's best performance. Referring to FIG. 14E, an individual multiple event analysis report 2050 includes performance, comment, and analysis data for the selected individual athlete 42. Specifically, the analysis data includes rank among a selected peer group for each event, automatically normalized performance data based on a correction for nonstandard environmental conditions for the 100 meter and 200 meter event, specifically wind conditions, and manually entered comment data based on nonstandard environmental conditions for the ¾ mile event, specifically, a thick grass surface and unconfirmed distance.

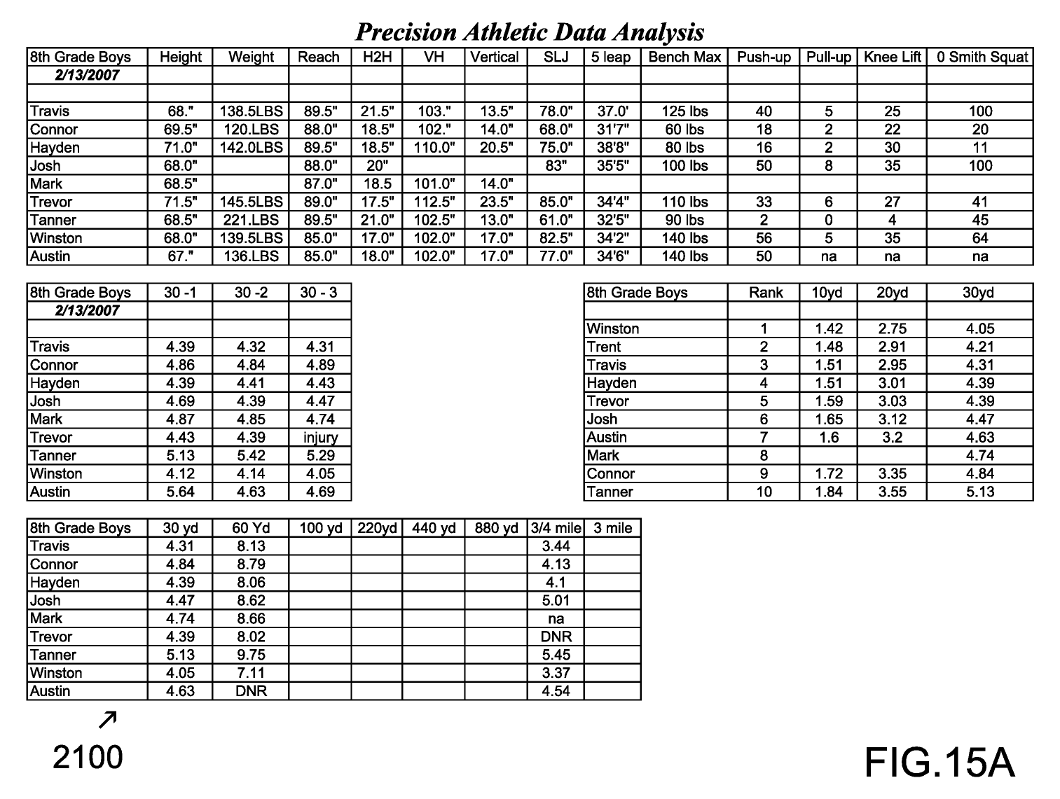

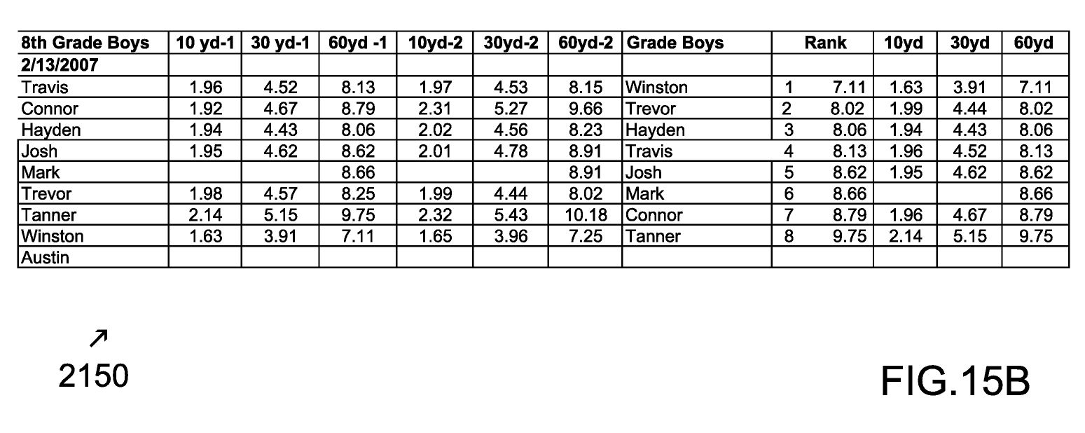

Referring to FIG. 15A, a first collective multiple event analysis reports 2100 includes demographic, body menu, performance, comment, and analysis data for a selected group of athletes 42 and a selected group of standardized test. Specifically, the comment data includes a reported injury resulting in no recorded performance for a particular trial of one test, and the analysis data includes ranking of the selected group for a selected test, the 30 yard dash. The report 2100, and other reports may additionally or alternative include information relating to different groups or teams, for example, multiple teams competing in a track and field meet. Referring to FIG. 15B, a collective single event analysis report 2150 includes performance and analysis data for a selected group of athletes 42 and multiple trials of a selected standardized test, specifically the 60 yard dash.

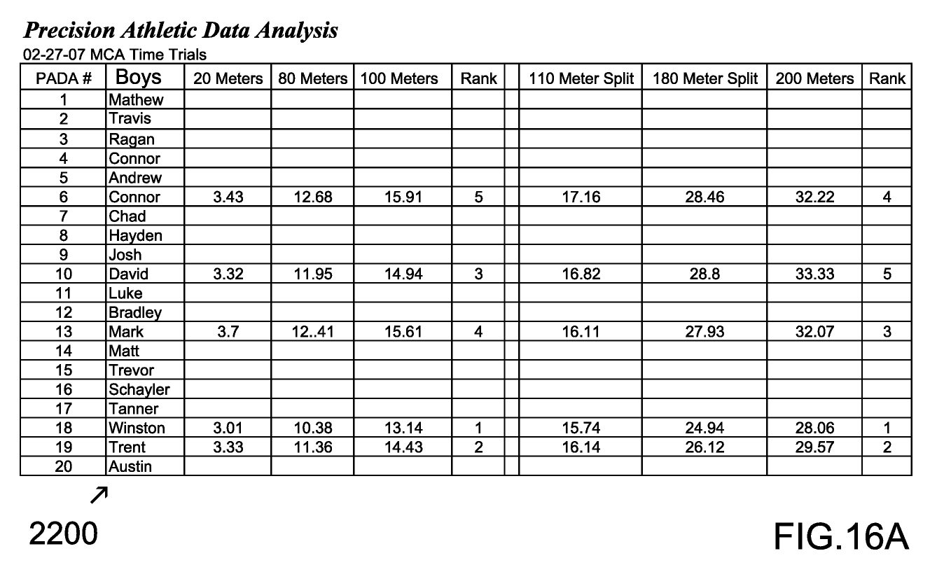

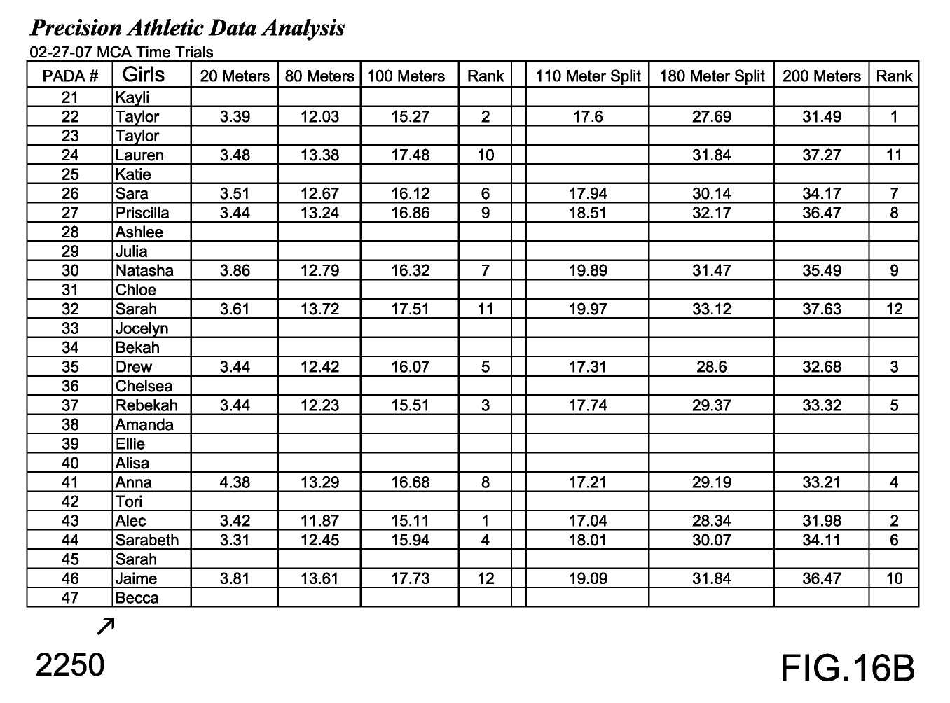

Referring to FIG. 16A, a second collective multiple event analysis reports 2200 includes performance and analysis data for a first selected group of athletes and a selected group of standardized tests, specifically the 100 meter and 200 meter dash. The performance data includes split times and the analysis data includes ranking for each event. Referring to FIG. 16B, a third collective multiple event analysis report 2250 includes performance and analysis data for a second selected group of athletes and the same selected group of standardized tests as for report 2200.

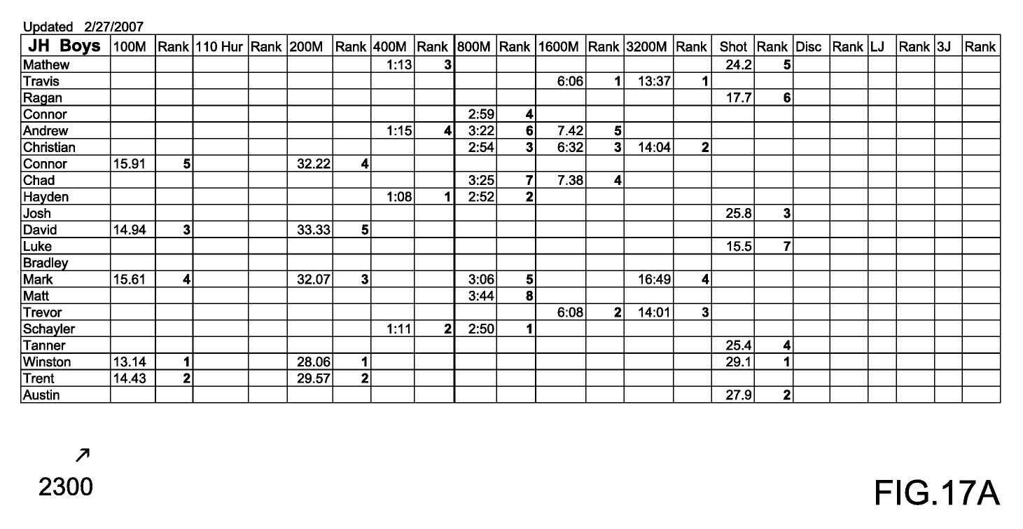

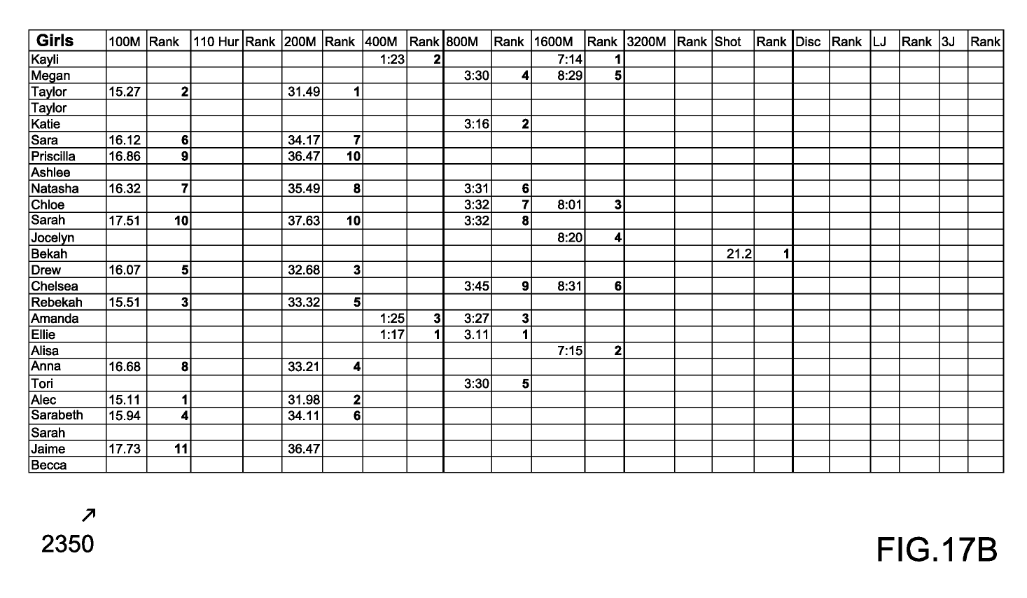

Referring to FIG. 17A, a fourth collective multiple event analysis report 2300 includes performance and analysis data for the first selected group of athletes and a different selected group of standardized tests. Referring to FIG. 17B, a fifth collective multiple event analysis report 2350 includes performance and analysis data for the second selected group of athletes 42 and the same selected standardized tests as for report 2300.

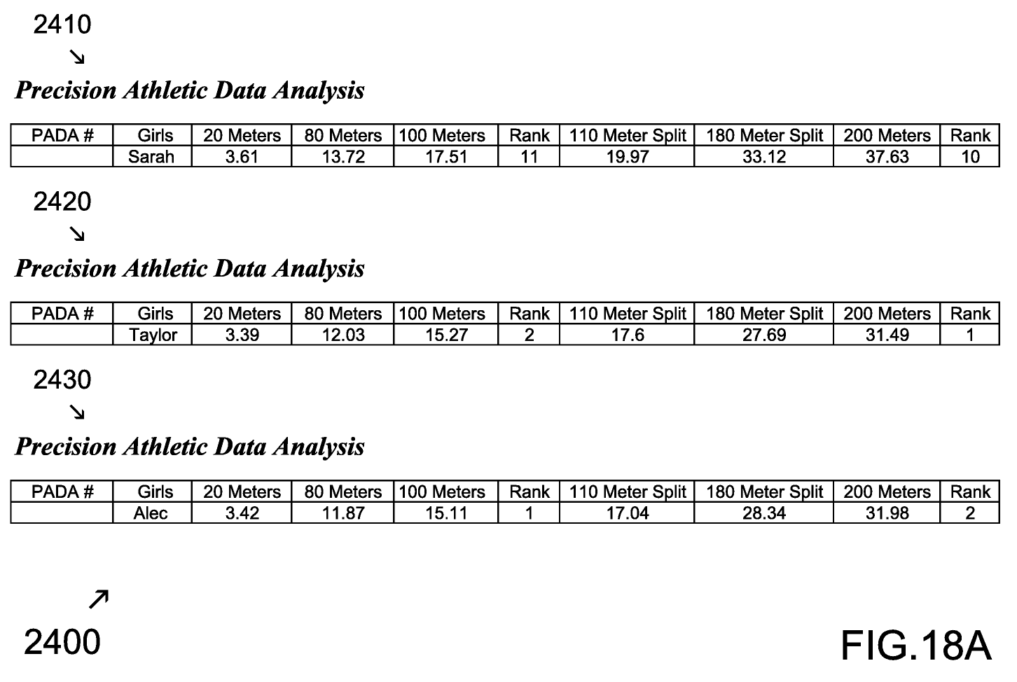

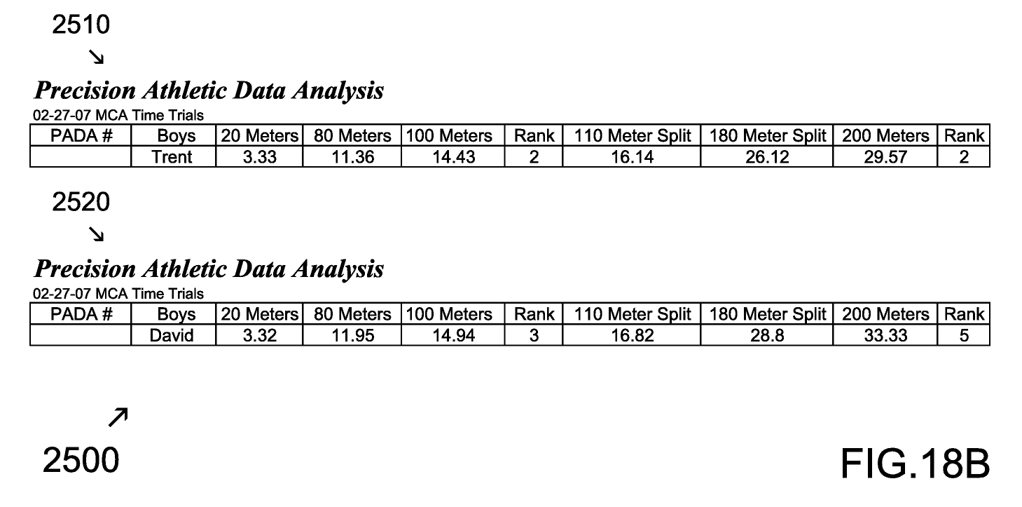

A first athlete comparison analysis report 2400 shown in FIG. 18A includes performance and analysis data for a first selected group of athletes 2410, 2420, and 2430 chosen for comparison. Specifically, report 2400 includes split and final times and ranking analysis for the 100 meter and 200 meter standardized tests. Similarly, a second athlete comparison analysis report 2500 shown in FIG. 18B includes performance and analysis data for a second selected group of athletes 2510 and 2520 chosen for comparison. The predication can be based at least in part on demographic, performance, training, and other data associated with the athlete, and data associated with another comparable athlete or data based on statistical analysis of multiple athletes. The prediction can be based solely on an athlete's change in maturity, or alternatively, based on changes in training, body metrics, or other data associated with the athlete.

While the invention has been illustrated and described in detail in the foregoing drawings and description, the same is to be considered as illustrative and not restrictive in character, it being understood that only illustrative embodiments thereof have been shown and described and that all changes and modifications that come within the spirit and scope of the invention as defined in the following claims are desired to be protected. For example, while the illustrative system 40 has been described in the context of athletics, all the features of the system 40 are also applicable to other areas of human evaluation, training, and performance, including physical therapy, health care, and rehabilitation.

1. A system for collecting human performance data, comprising:
a first performance sensor;
a locator device adapted for determining an actual location of the first performance sensor and for providing a location signal;
preset location parameters;
a data processor adapted to compare the location and the preset parameters and to output at least one of a plurality of signals based on the comparisons.

2. The system of claim 1, further comprising:
a first signal provided by the data processor upon the actual location satisfying the preset location parameters; and
an indicator adapted to receive and respond to the first signals.

3. The system of claim 1, wherein the preset location includes a preset displacement and the actual location includes a relative displacement between the first performance sensor and a reference point.

4. The system of claim 3, wherein the plurality of signals includes:
a first signal provided by the data processor upon the relative displacement satisfying the preset displacement parameters;
a second signal provided by the data processor upon the relative displacement being less than the preset displacement parameters;
a third signal provided by the data processor upon the relative displacement being greater than the preset displacement parameters; and
an indicator, the indicator adapted to display a different indication for each of the first, second, and third signals.

5. The system of claim 1, further comprising:
an environmental sensor adapted to provide an environmental measurement;
preset environmental parameters associated with the environmental measurement; and
a certification signal provided by the data processor upon the environmental measurement satisfying the preset environmental parameters and the actual location satisfying the present location parameters.

6. The system of claim 1, further comprising:
a second performance sensor, and
wherein the preset and actual location include a displacement between the first and second performance sensors.

7. The system of claim 1, wherein the locator device includes a distance measuring laser.

8. The system of claim 1, wherein the locator device includes a GPS receiver.

9. The system of claim 1, further comprising a video camera for capturing image data, and wherein the data processor is further adapted to time associate the image data and human performance data.

10. The system of claim 1, further comprising a geographically remotely located database for storing the human performance data.

11. The system of claim 1, further comprising:
a first data structure including data fields for storing human performance data associated with a first person and the first performance sensor; and
a second data structure including data fields for storing human performance data associated with at least a second person and spanning a first period of development; and
wherein the data processor is further adapted to determine a predicted human performance of the first person over a second period of development based at least in part on the human performance data associated with the at least a second person.

12. A system for collecting human performance data, comprising:
a performance sensor;
an environmental sensor adapted to provide an environmental measurement;
preset environmental parameters associated with the environmental data;
a data processor adapted to compare the preset environmental parameters with the environmental data, the data processor further adapted to output a signal based on the comparison.

13. The system of claim 12, further comprising a certification signal provided by the data processor upon the environmental measurement satisfying the preset environmental parameters.

14. The system of claim 12, wherein the data processor is further adapted to normalize the human performance data based on the environmental measurement.

15. The system of claim 14, further comprising a geographically remotely located database storing normalization data and algorithms, and wherein the normalization based on the environmental measurement receives and uses at least one of normalization data and algorithms.

16. The system of claim 12, further comprising:
a first data structure including data fields for storing human performance data associated with a first person and the performance sensor; and
a second data structure including data fields for storing human performance data associated with at least a second person and spanning a first period of development; and
wherein the data processor is further adapted to determine a predicted human performance of the first person over a second period of development based at least in part on the human performance data associated with the at least a second person.

17. A system for collecting and analyzing human performance data, comprising:
a field device for collecting human performance data;
a first data structure associated with the field device, the first data structure including data fields for storing human performance data associated with a first person;
a database adapted to be accessed by the field device;
a second data structure associated with the database, the second data structure including data fields for storing human performance data associated with at least a second person and spanning a first period of development; and
a data processor adapted to determine a predicted human performance of the first person over a second period of development based at least in part on the human performance data associated with the at least a second person.

18. The system of claim 17, wherein the database is geographically remotely located relative to the field device.

19. The system of claim 17, wherein the at least a second person includes an elite athlete.

20. The system of claim 17, wherein the predicted human performance includes a measure of a specific human performance associated with a specific sport position.

21. The system of claim 17, wherein the first data structure includes a body metric associated with the first person and wherein the predicted human performance is further based on a selected change in the body metric.

22. The system of claim 17, wherein the first data structure includes a training regime associated with the first person and wherein the predicted human performance is further based on a selected change in the training regime.

23. The system of claim 17, further comprising:
an environmental sensor for measuring environmental data; and
a preset environmental parameter associated with the measured environmental data, and
wherein the data associated within the first data structure is transmitted to the database and associated with the second data structure upon the measured environmental data satisfying the preset environmental parameter.
